(19) United States
(12) Patent Application Publication
(10) Pub. No.: US 2026/0234683 A1
(43) Pub. Date: Aug. 13, 2026

(54) MANUFACTURE OF MESSENGER RNA WITH KP34 POLYMERASE

(71) Applicant: SANOFI PASTEUR INC., Swiftwater, PA (US)

(72) Inventors: Jianping CUI, Cambridge, MA (US); Yaroslav MOROZOV, Cambridge, MA (US)

(21) Appl. No.: 19/160,680

(22) PCT Filed: Mar. 7, 2024
(86) PCT No.: PCT/EP2024/056094

§ 371 Date: Aug. 28, 2025

(30) Foreign Application Priority Data
Mar. 7, 2023 (EP) 23305304.0

(51) Int. Cl. C12P 19/34; C12N 9/12

(52) U.S. Cl. CPC C12P 19/34; C12N 9/1247; C12Y 207/07006

(57) ABSTRACT

The present disclosure provides methods and compositions for improving the in vitro transcription (IVT) of messenger RNA using Klebsiella phage KP34 RNA polymerase. In particular, the present disclosure provides a method for manufacturing messenger RNA comprising: (a) providing a DNA template comprising a nucleic acid sequence encoding an mRNA transcript for expression of a polypeptide or protein; (b) contacting the DNA template with a Klebsiella phage KP34 RNA polymerase under conditions suitable for IVT of the mRNA transcript.

### SEQUENCE LISTING

The present specification makes reference to a Sequence Listing (submitted electronically as an .xml file name “2024 Mar. 7 PAT23003-WO-PCT Sequence Listing” on 7 Mar. 2024. The .xml file was generated on 7 Mar. 2024 and is 47 KB in size. The entire contents of the sequence listing are herein incorporated by reference.

### FIELD OF THE INVENTION

The present invention generally relates to methods and compositions for improving the in vitro transcription (IVT) of messenger RNA (mRNA). In particular, the invention relates to the use of Klebsiella phage KP34 polymerase in the manufacture of mRNA.

### BACKGROUND OF THE INVENTION

Messenger RNA (mRNA) is becoming increasingly important as a therapeutic agent. mRNA therapy can be used to restore normal levels of an endogenous protein or provide an exogenous therapeutic protein (e.g., a vaccine antigen or antibody) without permanently altering the genome sequence or entering the nucleus of the cell. mRNA therapy takes advantage of the cell's own protein production and processing machinery to express a therapeutic peptide, polypeptide, or protein, is flexible to tailored dosing and formulation, and is broadly applicable to any disease or condition that is treatable through the provision of an exogenous protein.

The process of manufacturing mRNA for use in therapy typically involves the in vitro transcription (IVT) of mRNA from a DNA template using a phage-derived DNA-dependent RNA polymerase. This synthesis process commonly yields transcriptional by-products in addition to the desired mRNA transcripts. For example, T7 RNA polymerase forms double-stranded RNA (dsRNA) during IVT. Double-stranded RNA (dsRNA) is undesirable since it leads to inefficient translation of the administered mRNA product and results in the induction of cytokines, eliciting an interferon (IFN)-mediated inflammatory immune response.

For example, activation of dsRNA-dependent enzymes, such as oligoadenylate synthetase (OAS), RNA-specific adenosine deaminase (ADAR), and RNA-activated protein kinase (PKR), can trigger a cellular response resulting in the inhibition of protein synthesis, reducing the efficiency of mRNA therapy. dsRNA may also stimulate cellular pathogen sensors, e.g., Toll-like receptor 3 (TLR3), retinoic acid-inducible gene I (RIG-I), and melanoma differentiation-associated protein 5 (MDA5), leading to the secretion of different cytokines, including type I interferons, interleukin-6 (IL-6), and tumor necrosis factor-α (TNF-α).

As dsRNA is highly immunogenic, this creates impediments to using mRNA that contains dsRNA contaminants. Accordingly, it is desirable to eliminate or greatly reduce the amount of dsRNA from the IVT mRNA for many reasons including, for example, to limit cytokine induction and reduction of protein synthesis.

Karikó et al. (Nucleic Acids Res. 2011; 39 (21): e142) used high performance liquid chromatography (HPLC) to remove dsRNA from mRNA transcripts prepared by IVT using T7 polymerase. In in vitro transfection experiments, HPLC-purified mRNA was up to 1000-fold more potent in inducing expression of the mRNA-encoded protein. However, HPLC-based purification cannot easily be scaled, is expensive, and often requires the use of toxic organic solvents that need to be subsequently removed by additional purification steps. Accordingly, efforts have been made to develop alternative purification methods. For example, Baiersdörfer et al. (Mol Ther Nucleic Acids. 2019; 15:26-35) describe a cellulose-based chromatography method of removing dsRNA contaminants. This method avoids the use of toxic organic solvents, is more cost-effective, and does not face the same scalability issues as prior HPLC-based methods. Using higher ethanol concentrations further improves the efficiency of dsRNA removal when using the cellulose-based method. However, the recovery of single-stranded in vitro transcribed mRNA is reduced at these concentrations (Kwon et al., Arch Pharm Res. 2022; 45 (4): 245-262).

To avoid time-consuming and expensive purification of the mRNA after IVT, efforts have focused on reducing the formation of transcriptional by-products such as dsRNA. For example, SP6 RNA polymerase produces less dsRNA than T7 RNA polymerase (see, e.g., WO 2022/082001 and WO 2018/157153). Recently, DNA-dependent RNA polymerases have been discovered that are phylogenetically distant to T7 and SP6 (Xia et al., RNA Biol. 2022; 19 (1): 1130-1142). These enzymes produce much lower amounts of dsRNA than T7 and SP6 RNA polymerase during IVT of short RNA transcripts (less than 100 bases), but often at a reduced yield relative to T7 and SP6.

Accordingly, a need exists for methods that can be used at scale to manufacture mRNAs for the expression of therapeutic polypeptides or proteins (typically exceeding 500 bases in length), while producing only negligible amounts of transcriptional by-products.

### SUMMARY OF THE INVENTION

The present invention is based on the discovery that purified Klebsiella phage KP34 RNA polymerase can be employed to produce mRNA in place of the commonly used RNA polymerases, T7 and SP6. One stumbling block to using this polymerase for mRNA production has been the low mRNA yield. The inventors found that Klebsiella phage KP34 RNA polymerase expressed recombinantly in bacterial cells forms enzymatically inactive aggregates. Removing these aggregates or avoiding their formation in the first place (e.g., through genetic engineering) can dramatically improve the yield of long RNA transcripts (≥500 ribonucleotides) encoding polypeptides and proteins during IVT. The inventors also found that optimizing the nucleic acid sequence 3′ adjacent to the KP34 core promoter can further improve mRNA yield during in vitro synthesis, reaching levels comparable to SP6. Moreover, in comparison to T7 and SP6, Klebsiella phage KP34 RNA polymerase produces very few transcriptional by-products such as dsRNA. This makes Klebsiella phage KP34 RNA polymerase especially suitable for manufacturing mRNA for therapeutic uses.

Accordingly, the invention relates to a method for manufacturing mRNA comprising (a) providing a DNA template comprising a nucleic acid sequence encoding an mRNA transcript for expression of a polypeptide or protein, and (b) contacting the DNA template with a Klebsiella phage KP34 RNA polymerase under conditions suitable for IVT of the mRNA transcript. In some embodiments, the mRNA transcript is for expression of a therapeutic polypeptide or protein (e.g., for therapeutic use).

In particular embodiments, the Klebsiella phage KP34 RNA polymerase provided in step (b) is recombinantly expressed in Escherichia coli (E. coli) cells and purified to remove enzymatically inactive aggregates. In some embodiments, enzymatically inactive aggregates are removed from an affinity-purified preparation comprising the Klebsiella phage KP34 RNA polymerase. In some embodiments, the enzymatically inactive aggregates are removed by size exclusion chromatography (e.g., gel filtration). In some embodiments, the Klebsiella phage KP34 RNA polymerase comprises less than 5% enzymatically inactive aggregates (e.g., less than 4%, less than 3%, less than 2%, or less than 1%).

In some embodiments, the amino acid sequence of the Klebsiella phage KP34 RNA polymerase is at least 90% identical (e.g., at least 95%, at least 96%, at least 97%, at least 98%, at least 99% identical, or identical) to the amino acid sequence of SEQ ID NO: 1. In some embodiments, the Klebsiella phage KP34 RNA polymerase is present at a concentration ranging from 0.01 to 0.5 mg/mL. In some embodiments, the mRNA transcript comprises at least 500 ribonucleotides, e.g., at least 600, 700, 800, 900, or 1000 ribonucleotides.

In some embodiments, the amount of dsRNA comprised in the mRNA transcripts obtained in step (b) is below the limit of detection. The presence of dsRNA produced during IVT can be assessed with a dot blot assay using an anti-dsRNA monoclonal antibody (mAb), e.g., J2 mAb, K1 mAb, or K2 mAb. In particular embodiments, the dot blot assay uses J2 mAb. Alternatively, the presence of dsRNA produced during IVT can be assessed by ELISA using anti-dsRNA mAbs, e.g., J2 and K1 mAbs, or K1 and K2 mAbs. In particular embodiments, the ELISA uses J2 and K1 mAbs.

In some embodiments, the amount of dsRNA generated in step (b) is at least 10-fold lower (e.g., 20-fold, 50-fold or 100-fold) relative to a corresponding method using SP6 RNA polymerase or T7 RNA polymerase in place of the Klebsiella phage KP34 RNA polymerase. The amount of dsRNA can be determined by ELISA using anti-dsRNA mAbs, e.g., J2 and K1 mAbs or K1 and K2 mAbs, typically J2 and K1 mAbs. In some embodiments, the amount of dsRNA is determined by dot blot assay using an anti-dsRNA, e.g., J2 mAb, K1 mAb, or K2 mAb, typically J2 mAb.

In some embodiments, 10% by weight or less of the mRNA transcripts obtained in step (b) comprise non-templated nucleic acids. In some embodiments, the mRNA transcripts obtained in step (b) comprise less than 10% of dsRNA by weight (e.g., less than 5%, less than 3%, or less than 1%). In some embodiments, the amount of dsRNA is determined by ELISA using antibodies J2 and K1, or K1 and K2, typically J2 and K1.

In some embodiments, less than 10% by weight of the mRNA transcripts obtained in step (b) are abortive transcripts. In some embodiments, the abortive transcripts comprise less than 20 nucleotides. For example, in some embodiments, the abortive transcripts have a length of 2 to 19 nucleotides, e.g., between 5-15 nucleotides. In some embodiments, the abortive transcripts are detectable by gel electrophoresis.

In some embodiments, the method comprises synthesizing at least 1 mg of mRNA in a single batch (e.g., at least 10 mg, at least 100 mg, or at least 1 g).

In some embodiments, the DNA template comprises a Klebsiella phage KP34 promoter sequence operably linked to the nucleic acid sequence encoding the mRNA transcript. In some embodiments, the promoter sequence comprises the nucleic acid sequence set forth in SEQ ID NO: 5.

In some embodiments, the promoter sequence is optimized to improve mRNA transcript yield. In some embodiments, the yield of the mRNA transcripts obtained in step (b) is comparable to the yield achieved with a corresponding method using SP6 RNA polymerase or T7 RNA polymerase in place of the Klebsiella phage KP34 RNA polymerase. In some embodiments, the promoter sequence comprises the nucleic acid sequence set forth in SEQ ID NO: 7, 8, 9, or 41. In some embodiments, the promoter sequence comprises the nucleic acid sequence set forth in SEQ ID NO: 7 or 8. In some embodiments, the promoter sequence comprises the nucleic acid sequence set forth in SEQ ID NO: 19 or 20. In some embodiments, the promoter sequence comprises the nucleic acid sequence set forth in SEQ ID NO: 26, 32, or 35, e.g., SEQ ID NO: 26.

In some embodiments, the DNA template is at a concentration of 0.05 mg/mL to 0.5 mg/mL. In some embodiments, the DNA template is linear or linearized.

In some embodiments, IVT takes place in the presence of magnesium chloride (MgCl₂). In some embodiments, the MgCl₂ concentration is greater than 20 mM. In some embodiments, the MgCl₂ concentration is about 25 mM.

In some embodiments, IVT takes place in the presence of sodium chloride (NaCl). In some embodiments, the NaCl concentration is less than 20 mM. In some embodiments, the NaCl concentration is about 0.5 mM.

In some embodiments, IVT takes place in the presence of buffering agent. In some embodiments, the buffering agent is selected from Tris, HEPES, ammonium sulfate, sodium bicarbonate, sodium citrate, sodium acetate, potassium phosphate, and sodium phosphate. In some embodiments, the buffering agent is Tris-HCl. In some embodiments, Tris-HCl is present at a concentration of less than 40 mM. In some embodiments, Tris-HCl is present at a concentration of about 25 mM.

In some embodiments, IVT takes place in the presence of unmodified ribonucleotides. In some embodiments, IVT takes place in the presence of a modified ribonucleotide.

In some embodiments, the modified ribonucleotide has a modified nucleoside. In some embodiments, the modified nucleoside is selected from 2-aminoadenosine, 2-thiothymidine, inosine, pyrrolo-pyrimidine, 3-methyl adenosine, 5-methylcytidine, C-5 propynyl-cytidine, C-5 propynyl-uridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, pseudouridine, 2-thiouridine, and 2-thiocytidine. In some embodiments, the modified nucleoside is pseudouridine, N1-methylpseudouridine, 5-methylcytidine, or 5-methoxyuridine, typically N1-methylpseudouridine.

In some embodiments, IVT takes place in the presence of ribonucleotides, wherein each ribonucleotide is present at a concentration of 0.1 mM to 10 mM.

In some embodiments, IVT takes place at a pH of 7.0 to 7.7. In some embodiments, the pH is about 7.5.

In some embodiments, IVT takes place at a temperature of 37° C. to 42° C. In some embodiments, the temperature is 37° C.

In some embodiments, the IVT reaction takes place over a period of 30 minutes to 6 hours.

In some embodiments, IVT is terminated by addition of DNase I and a DNase I buffer.

In some embodiments, a method for manufacturing mRNA in accordance with the invention further comprises a step of purifying the mRNA transcripts obtained in step (b) from the Klebsiella phage KP34 RNA polymerase (and optionally other reactants and enzymes present after termination of IVT). In some embodiments, the step of purifying the mRNA transcripts involves a method other than (i) cellulose chromatography, and/or (ii) HPLC with a buffer system comprising triethylammonium acetate and/or acetonitrile.

The invention also relates to compositions obtainable by the methods for manufacturing mRNA disclosed herein. For example, the invention provides a composition comprising mRNA transcripts for expression of a polypeptide or protein and Klebsiella phage KP34 RNA polymerase, wherein the composition comprises less than 1% of dsRNA by weight and wherein less than 10% of the mRNA transcripts by weight are abortive transcripts.

The invention also includes mRNA that is obtained using a method of the present invention. Such mRNA may be distinguishable from prior art mRNA preparations in that it is free from residual cellulose or organic solvent (i.e., components used in the purification of IVT mRNA produced by other RNA polymerases such as T7). Such mRNA may also comprise substantially lower amounts of contaminating transcriptional by-products e.g., dsRNA, or other by-products of IVT that may be difficult to remove by purification.

The invention also relates to pharmaceutical compositions comprising mRNA obtained by the methods for manufacturing mRNA disclosed herein (in particular, mRNA encoding a therapeutic polypeptide or protein) and their therapeutic use, e.g., in a method of treating or preventing a disease or disorder in a subject.

Other features, objects, and advantages of the present invention are apparent in the detailed description, drawings and embodiments that follow. It should be understood, however, that the detailed description, the drawings, and the embodiments, while indicating embodiments of the present invention, are given by way of illustration only, not limitation. Various changes and modifications will become apparent to those skilled in the art.

### BRIEF DESCRIPTION OF THE DRAWINGS

Embodiments of the invention will be described, by way of example, with reference to the following drawings.

FIGS. 1A and 1B illustrate affinity-based purification of Klebsiella phage KP34 RNA polymerase. FIG. 1A shows an SDS-PAGE gel. From left to right, the following samples were loaded on the gel: E. coli whole cell lysate (“Lysate”), pelleted fraction resuspended in loading buffer for IMAC (“Ni load”), the flowthrough collected after loading (“Ni FT”), the fraction collected after washing the loaded IMAC column (“Ni Wash”), and various eluate fractions collected by applying the elution buffer with increasing imidazole concentration (“Ni Eluates” labelled as 1A6, 1A7, 1A8, 1A9, 1A10, 1A11, 1A12), a SeeBlue™ pre-stained protein standard (molecular weights of the protein standard are provided in kDa to the right of the gel image). The band corresponding to Klebsiella phage KP34 RNA polymerase (“KP34”) is marked by an arrow. FIG. 1B shows a chromatogram of the effluent from a Superdex™ 75 gel filtration column. UV absorbance at 280 nm was detected. The elution volume (in mL) is plotted against UV adsorption (in mAU). Elution with a buffer comprising increasing imidazole concentrations is indicated by vertical lines at the bottom of the graph. The major peak corresponding to purified KP34 is indicated by an arrow.

FIG. 2 illustrates that RNA-dependent 3′ end extension of mRNA transcripts does not occur when Klebsiella phage KP34 RNA polymerase is used for in vitro transcription (IVT). The T7, SP6 and KP34 RNA polymerases (RNAP) were tested in an RNA-dependent RNA polymerase (RdRp) assay at concentrations of 0 μM (negative control), 0.1 μM, 0.2 μM, or 0.6 μM. The polymerases were incubated with 0.4 μM of a 50 base long synthetic RNA (RNA50) as a template. This template was allowed to self-anneal. Annealing of an internal region of complementarity results in the formation of a stretch of dsRNA in cis by looping, enabling self-templated 3′end extension. The template was incubated in the presence of unmodified ATP, CTP, GTP, and UTP as described in Example 5. After incubation, each sample was gel separated to determine whether 3′ end extension had occurred. RNA was detected using SYBR Gold staining. The first lane, labelled ‘M’, corresponds to the molecular weight ladder, with molecular weights indicated in nucleotides (nt) to the left of the gel image. A representative band corresponding to RNA50 is marked with a line. A representative band comprising 3′ end extended RNAs is framed by two lines.

FIG. 3 illustrates that comparable mRNA yields are obtained from IVT reactions performed using either SP6 or Klebsiella phage KP34 RNA polymerase. Four DNA templates were tested (labelled as Sequence 2, 3, 4 or 5). The templates ranged in size from about 1100 bp to about 4700 bp. A control DNA template (labelled as Sequence 1 on FIG. 3) corresponds to the DNA template no. 1 recited in Example 3. Mean IVT yield from SP6 (left bar) and KP34 (right bar) for each sequence tested is shown. Error bars depict standard deviation.

FIG. 4 illustrates that IVT using Klebsiella phage KP34 RNA polymerase yields amounts of dsRNA undetectable by dot blot. IVT was performed with SP6 RNA polymerase (“SP6”) or Klebsiella phage KP34 RNA polymerase (“KP34”) in the presence of unmodified ribonucleotides or in the presence of a modified ribonucleotide as described in Example 2. The resulting mRNA transcripts are labelled accordingly as unmodified or modified mRNA (indicated as “Unmod” or “Mod”). 100 ng, 200 ng, or 400 ng RNA in a 2 μL sample volume was blotted on a nitrocellulose membrane. 1 ng, 20 ng, or 40 ng of dsRNA control was used as a reference. The anti-dsRNA monoclonal antibody J2 was used as the primary antibody. An anti-mouse IgG HRP was used as secondary antibody. Signal was detected after a one-minute exposure. The amount of dsRNA in samples prepared with Klebsiella phage KP34 RNA polymerase was many times lower relative to the amount of dsRNA produced when SP6 RNA polymerase was used for IVT under identical conditions.

FIGS. 5A and 5B illustrate the removal of enzymatically inactive aggregates from affinity-based purification of Klebsiella phage KP34 RNA polymerase. FIG. 5A shows a chromatogram of the effluent from a Superdex™ 200 gel filtration column. UV absorbance was detected at 280 nm (light grey, dashed line) and 400 nm (dark grey, solid line). The elution volume (in mL) is plotted against UV adsorption (in mAU). Two peaks, labelled as I and II, were identified. Both peaks corresponded to purified Klebsiella phage KP34 RNA polymerase. The peak I fraction eluted earlier, consistent with a greater particle size than the peak II fraction. Collected eluate fractions are indicated by vertical lines at the bottom of the graph and were analyzed by SDS-PAGE. FIG. 5B shows a representative SDS-PAGE gel, confirming that both the peak I fraction and the peak II fraction included Klebsiella phage KP34 RNA polymerase. From left to right, the following samples were loaded on the gel: a SeeBlue™ pre-stained protein standard (molecular weights of the protein standard are shown in kDa to the left of the gel image), the unpurified sample loaded onto the gel filtration column (“SEC Load”), and various eluate fractions collected during the gel filtration process (“SEC 1A9”, SEC 1A11″, “SEC 1B1”, “SEC 1B3”, “SEC 1B5”, “SEC 1C2”, “SEC 1C4”, “SEC 1C6”, “SEC 1C8”, “SEC 1C10”). The eluate fractions framed by a black solid border correspond to peak I fractions. The eluate fractions framed by a dashed border correspond to peak II fractions. The band corresponding to KP34 is marked by an arrow.

FIG. 6 illustrates the amounts of abortive transcripts that are formed by a purified Klebsiella phage KP34 RNA polymerase during IVT reactions with DNA templates comprising different promoter sequences. The bars represent the amount of peak area per 1 μg of the sample that was analyzed by liquid chromatography-mass spectroscopy (LC-MS) as described in Example 11. The value is the sum of all peaks between 6.5 and 12 minutes from duplicate samples. The sample IDs correspond to those in Table 11. For comparison, the amounts of abortive transcripts formed by purified SP6 and T7 RNA polymerases during IVT was also measured (labelled accordingly as “SP6” and “T7”). DNA template comprising the optimized KP34 promoter sequence of SEQ ID NO: 26 or SEQ ID NO: 35 (sample IDs 4 and 9) notably showed low levels of short abortive transcripts in IVT.

FIGS. 7A and 7B illustrate the results from a solubility tag screen designed to improve expression and solubility of Klebsiella phage KP34 RNA polymerase during recombinant expression in Escherichia coli (E. coli). In FIG. 7A, each bar represents the protein concentration (mg/mL) of a fusion protein comprising Klebsiella phage KP34 RNA polymerase and the indicated tag, purified using immobilized metal ion affinity chromatography (IMAC) and desalted. Fusion proteins labelled with * and ** yielded statistically significantly higher concentrations (p-value of 0.05 and <0.05, respectively) relative to the wild-type KP34 enzyme (WT). Level of WT protein is indicated as a dashed line. For comparison, an SP6 RNA polymerase was expressed and purified using the same process. Fusion proteins resulting in significantly higher yields were tested for their solubility relative to WT, as summarized in the bar graph shown in FIG. 7B. The fold increase in solubility relative to WT is indicated in each bar. Highly expressed fusion proteins had greater solubility relative to WT.

FIG. 8 illustrates the results of an in vitro transcription (IVT) reaction using each of the polymerase fusion proteins tested in the solubility tag screen shown in FIG. 7A. The IVT reactions were performed as described in Example 3. The resulting RNA concentrations are shown in ng/μl. Despite normalization of the protein concentration prior to the IVT assay, fusion proteins that were expressed at significantly higher levels and that had improved solubility relative to WT also showed a trend towards yielding more mRNA per reaction.

### DEFINITIONS

In order for the present invention to be more readily understood, certain terms are first defined below. Additional definitions for the following terms and other terms are set forth throughout the specification.

As used in this specification and the appended claims, the singular forms “a”, “an”, and “the” include plural referents unless the context clearly dictates otherwise. For example, “a ribonucleotide” is understood to represent one or more ribonucleotides. As such, the terms “a” (or “an”), “one or more”, and “at least one” can be used interchangeably herein.

Unless specifically stated or obvious from context, as used herein, the term “or” is understood to be inclusive and covers both “or” and “and”. Furthermore, “and/or” where used herein is to be taken as specific disclosure of each of the two specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification and embodiments, the words “have” and “comprise”, or variations such as “has”, “having”, “comprises”, or “comprising” will be understood to imply the inclusion of a stated integer or group of integers but not the exclusion of any other integer or group of integers. It is further understood that wherever embodiments are described herein with the language “comprising” or “having” of grammatical equivalents thereof, otherwise analogous embodiments described in terms of “consisting of” and/or “consisting essentially of” are also provided.

As used herein, the term “about” refers to an interval of accuracy that a person skilled in the art will understand to still ensure the technical effect of the feature in question. The term indicates a deviation from the indicated numerical value of +10%. In some embodiments, the deviation is +5% of the indicated numerical value. In certain embodiments, the deviation is =1% of the indicated numerical value.

As used herein, the term “mRNA” refers to a polyribonucleotide that encodes at least one polypeptide. mRNA as used herein encompasses both modified and unmodified RNA. mRNA may contain one or more coding and non-coding regions (e.g., a 5′ untranslated region and a 3′ untranslated region). mRNA can be purified from natural sources, produced using recombinant expression systems and optionally purified, in vitro transcribed, or chemically synthesized. The invention particularly relates to in vitro transcribed mRNA. Where appropriate, e.g., in the case of chemically synthesized molecules, mRNA can comprise nucleoside analogues such as analogues having chemically modified bases or sugars, backbone modifications, etc. An mRNA sequence is presented in the 5′ to 3′ direction unless otherwise indicated. A typical mRNA comprises a 5′ cap, a 5′ untranslated region (5′ UTR), a protein-coding region, a 3′ untranslated region (3′ UTR), and a 3′ tail. In some embodiments, the tail structure is a poly(C) tail. More typically, the tail structure is a polyA tail.

As used herein, the terms “Klebsiella phage KP34 RNA polymerase”, “KP34 RNA polymerase”, “KP34 polymerase”, and “KP34” are used interchangeably and all refer to a DNA-dependent RNA polymerase obtainable from a Klebsiella phage (e.g., an RNA polymerase with the amino acid sequence set forth in SEQ ID NO: 1).

The term “naturally occurring” as used herein to describe a Klebsiella phage KP34 RNA polymerase amino acid sequence refers to the wild-type or native amino acid sequence. In some embodiments, the Klebsiella phage KP34 RNA polymerases disclosed herein may include one or more amino acid substitutions, deletions, insertions, and/or additions relative to the naturally occurring amino acid sequence in order to render the protein more suitable for use in the methods and compositions of the invention. Without wishing to be bound by any particular theory, the inventors believe that the polymerase function of such a modified enzyme has essentially identical or improved polymerase activity relative to the wild-type or native enzyme.

As used herein, the term “sequence-optimized” is used to describe a nucleotide sequence that is modified relative to a naturally occurring or wild-type nucleotide sequence. In the case of a sequence-optimized mRNA, such modifications may include, e.g., codon optimization and/or the use of 5′ UTRs and 3′ UTRs which are not normally associated with the naturally occurring or wild-type nucleic acid. As used herein, the terms “codon optimization” and “codon-optimized” refer to modifications of the codon composition of a naturally occurring or wild-type nucleic acid encoding a peptide, polypeptide, or protein that do not alter its amino acid sequence, thereby improving protein expression of said nucleic acid. In the context of the present invention, “codon optimization” may also refer to the process by which one or more optimized nucleotide sequences are arrived at by removing, with filters, less than optimal nucleotide sequences from a list of nucleotide sequences, such as filtering by guanine-cytosine (GC) content, codon adaptation index (CAI), presence of destabilizing nucleic acid sequences or motifs, and/or presence of pause sites and/or terminator signals.

As used herein, the term “template DNA” (or “DNA template”) refers to a DNA molecule comprising a nucleic acid sequence encoding an mRNA transcript to be synthesized by IVT. The template DNA is used as template for IVT in order to produce the mRNA transcript encoded by the template DNA. The template DNA comprises all elements necessary for IVT, particularly a promoter element for binding of a DNA-dependent RNA polymerase, which is operably linked to the DNA sequence encoding a desired mRNA transcript. Furthermore, the template DNA may comprise primer binding sites 5′ and/or 3′ of the DNA sequence encoding the mRNA transcript to determine the identity of the DNA sequence encoding the mRNA transcript, e.g., by PCR or DNA sequencing. The “template DNA” in the context of the present invention may be a linear or a circular DNA molecule. As used herein, the term “template DNA” may refer to a DNA vector, such as a plasmid DNA, which comprises a nucleic acid sequence encoding the desired mRNA transcript.

The terms “short abortive transcript” and “abortive transcript” are used interchangeably herein. They refer to transcripts that are generated during abortive initiation of transcription by an RNA polymerase. Abortive transcripts are commonly observed during an in vitro transcription (IVT) reaction. Abortive transcripts typically comprise less than about 20 nucleotides. During in vitro synthesis of mRNA, RNA polymerase (RNAP) recognizes its cognate promoter leading to the local melting of a double-strand DNA template to form the transcriptional “initiation complex”. Transcription during this stage is characterized by the repetitive synthesis and release of two to six nucleotides called “abortive cycling”, which is common to all RNAPs. Even at saturating nucleotide concentrations, abortive transcripts are present in reaction in vitro, although their lengths differ among different RNAPs. After the synthesis of about eight to twelve nucleotides, the polymerase undergoes a major structural rearrangement and dissociates from the promoter (promoter clearance) to enter the processive synthesis of RNA, forming the “elongation complex” until transcription termination. Since the initiation complex is unstable, when compared to the elongation complex, abortive transcripts are repeatedly released until the polymerase engages in productive transcription, which produces full-length transcripts.

As used herein, the term “truncated transcript” is used to refer to any transcript generated during the elongation phase that is shorter than a full-length mRNA molecule encoded by the DNA template, e.g., as a result of the premature termination of transcription. In some embodiments, a truncated transcript may be less than 90% of the length of the full-length mRNA molecule that is transcribed from the target molecule, e.g., less than 80%, 70%, 60%, 50%, 40%, 30%, 20%, or 10%. The length of an mRNA molecule that encodes a full-length polypeptide or protein is at least 90% of the length of the theoretical transcript length. Indeed, the theoretical transcript length may differ from the measured length using a specific assay. Accordingly, in some embodiments, the term “full-length mRNA” refers to the measured length as characterized when using a specific assay, e.g., gel electrophoresis and detection using UV, or UV absorption spectroscopy with separation by capillary gel electrophoresis. In embodiments that use such a specific assay, an mRNA transcript transcribed from a DNA template is considered full-length if it is at least 95% (e.g., at least 96%, 97%, 98%, or 99%) of the theoretical length of a corresponding reference mRNA that expresses the full-length polypeptide or protein encoded by the mRNA transcript.

As used herein, the term “double-stranded RNA” or “dsRNA” refers to RNA produced during IVT comprising two complementary strands of ribonucleic acids base-paired with each other. During IVT, dsRNA is generated in cis by looping of full-length RNA with internal regions of complementarity. In addition, abortive transcripts are generated during the initiation phase of IVT, and the 3′ end of the full-length RNA can prime complementary RNA synthesis from the primary transcripts in trans. Promoter-independent transcription of full-length anti-sense RNA is another mechanism of dsRNA generation.

As used herein, the term “batch” refers to a quantity or amount of mRNA synthesized at one time, e.g., produced according to a single manufacturing order during the same cycle of manufacture. A batch may refer to an amount of mRNA synthesized in one reaction that occurs via a single aliquot of enzyme and/or a single aliquot of DNA template for continuous synthesis under one set of conditions. In some embodiments, a batch would include the mRNA produced from a reaction in which not all reagents and/or components are supplemented and/or replenished as the reaction progresses. The term “batch” would not mean mRNA synthesized at different times that are combined to achieve the desired amount.

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein. In this application, the terms “expression” and “production,” and grammatical equivalents, are used interchangeably.

As used herein, the term “therapeutic” refers to any pharmaceutical, drug, or composition that can be used to treat or prevent a disease, illness, condition, or disorder of bodily function.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “in vitro” refers to events that occur in an artificial environment, e.g., in a test tube or reaction vessel, in cell culture, etc., rather than within a multi-cellular organism. For example, as used herein, the term “in vitro transcription” or “in vitro synthesis” refers to transcription or synthesis of RNA that occurs outside of a cell and/or in the absence of a cell lysate, typically in a test tube or reaction vessel (e.g., a bioreactor). “In vitro transcription” or “in vitro synthesis” typically involves the use of recombinantly produced and purified enzyme components (e.g., KP34 RNA polymerase).

As used herein, the term “in vivo” refers to events that occur within a multi-cellular organism, such as a human or a non-human animal. In the context of cell-based systems, the term may be used to refer to events that occur within a living cell (as opposed to, for example, in vitro systems).

As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man.

Unless otherwise defined herein, technical and scientific terms used herein have the same meaning as commonly understood by one of ordinary skill in the art to which this application belongs and as commonly used in the art to which this application belongs. Exemplary methods and materials are described below, although methods and materials similar or equivalent to those described herein can also be used in the practice or testing of the present disclosure. In case of conflict, the present specification, including definitions, will control.

Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular.

All publications and other reference materials referenced herein are hereby incorporated by reference in their entirety. Although a number of documents are cited herein, this citation does not constitute an admission that any of these documents forms part of the common general knowledge in the art.

### DETAILED DESCRIPTION OF THE INVENTION

The present invention relates to a method for manufacturing mRNA using Klebsiella phage KP34 RNA polymerase under conditions suitable for IVT of the mRNA transcript. The present invention is based, in part, on the discovery that Klebsiella phage KP34 RNA polymerase generates fewer transcriptional by-products e.g., dsRNA, than other RNA polymerases commonly used for IVT, e.g., T7 and SP6.

In particular, the present invention provides a method for manufacturing mRNA for therapeutic use comprising (a) providing a DNA template comprising a nucleic acid sequence encoding an mRNA transcript for expression of a therapeutic polypeptide or protein and (b) contacting the DNA template with a Klebsiella phage KP34 RNA polymerase under conditions suitable for IVT of the mRNA transcript.

### Klebsiella Phage KP34 RNA Polymerase

Klebsiella phage KP34 RNA polymerase is a DNA-dependent RNA polymerase that is only distantly related to other known phage-derived polymerases such as T7 and SP6.

Naturally occurring Klebsiella phage KP34 RNA polymerase (NCBI Reference Sequence: YP_003347629.1) has the following amino acid sequence:

(SEQ ID NO: 1)
MISALSTVVVPEEALVKRQLELEETYKIRGIERARKLITDALQNGGIMNLPMTQRM
LTSAYEVAAAAIDEMRNVKAPGIGGKYRRFLRLIPLDVLTTLSLCTMFEAFSVAPG
ESASRRQTAQAVMSALGRNVQSELLSLQLRNVAPAYMDRVYEYLTERRTKSPTHI
LRTLRASAENVHYGHEPWTNAQNISVGRLLCAAVFETGLFQWKTGSGNLSMLYPA
DDVMEAFQQLVESADTVTMKPPMLVPPVQHTTMWDGGYLTPIDNRGTYHNSHID
RTRLREVAEAFKSADGIKKALNKAQETPYRINKRILELVQEARALGIGVGMPRSVP
EPKPEWYLDGVPKENYTEEELDRFGEWKTRMSLWYSADRKRVSQLRSLLTTLEM
AEEFKDEKALYFPTCVDWRYRLYFKSSLHPQGSDLQKALLEFGRGKPLGDRGLFW
LKVHVATCFGYDKTLFEDRAAWVDANFAEIEQLTVSPFDCPAFTSADSPWCLLAA
AIDLVNAVRSGCPEEHISRIPVAMDATNSGGQHLSALLRDPVGGRLTNLYWEGND
KKADLYMDVKRRTDEKVILDLDKEDFIIQSTYWRENEITRSMTKRPSMTYFYSATV
RSCSDYIFEGACAEGYEGTDTNSLWNLSCYLAPRMRAAIEEANPAAAAVMGYLQN
LARRVPASQHLQWYTPLGGLVMNRYTQREEVRVRIDCMNLSAVLVHNRDFKTCN
KRKAASGIAPNFVHSLDSTHLMMVLCAAEGLDIVPIHDSLATHAADVDDMHRHIR
EQFVRLYEENDLLGDITRAAAAAGADLTDLDMPEVGTLDIRQVLESPFFFC.

A Klebsiella phage KP34 RNA polymerase suitable for use with the present invention can be a modified enzyme having substantially the same or improved polymerase activity as a naturally occurring Klebsiella phage KP34 RNA polymerase. Thus, in some embodiments, the KP34 RNA polymerase may be modified from SEQ ID NO: 1, e.g., may comprise one or more amino acid substitutions, deletions, insertions, and/or additions relative to SEQ ID NO: 1. In some embodiments, a suitable KP34 RNA polymerase has an amino acid sequence that is about 80%, 85%, 86%, 87%, 88%, 89%, 90%, 91%, 92%, 93%, 94%, 95%, 96%, 97%, 98%, or 99% identical to SEQ ID NO: 1. In particular embodiments, the amino acid sequence of the KP34 RNA polymerase is at least about 90% (e.g., at least about 91%, 92%, 93%, 94%, or 95%) identical to the amino acid sequence of SEQ ID NO: 1.

In some embodiments, a suitable KP34 RNA polymerase may be a truncated protein (from N-terminus, C-terminus, or internally) but retain the polymerase activity. In some embodiments, a suitable KP34 RNA polymerase is a fusion protein. For example, a KP34 RNA polymerase may include one or more tags to promote isolation, purification, or solubility of the enzyme. A suitable tag may be located at the N-terminus, C-terminus, and/or internally. Typically, the tag is located at the N-terminus. Non-limiting examples of a suitable tag include Calmodulin-binding protein (CBP); Fasciola hepatica 8-kDa antigen (Fh8); FLAG tag peptide; glutathione-S-transferase (GST); Histidine tag (e.g., hexahistidine tag (His6)); maltose-binding protein (MBP); N-utilization substance (NusA); small ubiquitin related modifier (SUMO) fusion tag; Streptavidin binding peptide (STREP); Tandem affinity purification (TAP); and thioredoxin (TrxA). Other tags may be used in the present invention. These and other fusion tags have been described, e.g., in Costa et al., Frontiers in Microbiology, 5 (2014): 63 and in PCT/US16/57044, the contents of which are incorporated herein by reference in their entireties. In some embodiments, a His tag is located at the N-terminus of KP34.

In a specific embodiment, a suitable Klebsiella phage KP34 RNA polymerase is modified for purification by affinity chromatography, as described, e.g., in Example 1. A suitably modified Klebsiella phage KP34 RNA polymerase for use with the invention has the following amino acid sequence:

(SEQ ID NO: 2)
MGHHHHHHGGISALSTVVVPEEALVKRQLELEETYKIRGIERARKLITDALQNGGI
MNLPMTQRMLTSAYEVAAAAIDEMRNVKAPGIGGKYRRFLRLIPLDVLTTLSLCT
MFEAFSVAPGESASRRQTAQAVMSALGRNVQSELLSLQLRNVAPAYMDRVYEYL
TERRTKSPTHILRTLRASAENVHYGHEPWTNAQNISVGRLLCAAVFETGLFQWKTG
SGNLSMLYPADDVMEAFQQLVESADTVTMKPPMLVPPVQHTTMWDGGYLTPIDN
RGTYHNSHIDRTRLREVAEAFKSADGIKKALNKAQETPYRINKRILELVQEARALGI
GVGMPRSVPEPKPEWYLDGVPKENYTEEELDRFGEWKTRMSLWYSADRKRVSQL
RSLLTTLEMAEEFKDEKALYFPTCVDWRYRLYFKSSLHPQGSDLQKALLEFGRGKP
LGDRGLFWLKVHVATCFGYDKTLFEDRAAWVDANFAEIEQLTVSPFDCPAFTSAD
SPWCLLAAAIDLVNAVRSGCPEEHISRIPVAMDATNSGGQHLSALLRDPVGGRLTN
LYWEGNDKKADLYMDVKRRTDEKVILDLDKEDFIIQSTYWRENEITRSMTKRPSM
TYFYSATVRSCSDYIFEGACAEGYEGTDTNSLWNLSCYLAPRMRAAIEEANPAAA
AVMGYLQNLARRVPASQHLQWYTPLGGLVMNRYTQREEVRVRIDCMNLSAVLV
HNRDFKTCNKRKAASGIAPNFVHSLDSTHLMMVLCAAEGLDIVPIHDSLATHAAD
VDDMHRHIREQFVRLYEENDLLGDITRAAAAAGADLTDLDMPEVGTLDIRQVLES
PFFFC.

In some embodiments, a Klebsiella phage KP34 RNA polymerase for use with a method of the invention can be prepared recombinantly as described in Example 1 using an expression plasmid comprising the following coding sequence, which is optimized for expression in E. coli:

(SEQ ID NO: 3)
ATGGGACATCACCATCACCATCACGGAGGCATTAGCGCCCTAAGTACGGTAGT
AGTACCAGAGGAAGCACTGGTGAAACGCCAGCTGGAGCTTGAAGAGACCTATA
AGATTCGCGGAATCGAGCGGGCACGTAAGCTGATTACGGACGCATTGCAGAAC
GGCGGGATTATGAACCTGCCTATGACGCAGCGTATGCTCACTTCGGCATATGAA
GTGGCTGCTGCCGCGATCGATGAGATGCGAAATGTCAAAGCCCCTGGCATTGG
TGGGAAGTATCGCCGGTTCCTGCGCTTAATCCCCTTGGATGTCCTGACCACCCT
GAGCCTGTGCACAATGTTTGAGGCGTTCAGCGTCGCCCCCGGCGAGTCCGCCA
GTCGCCGCCAGACTGCGCAGGCGGTAATGTCCGCACTGGGCCGGAACGTGCAG
TCGGAACTGCTGTCTTTGCAGCTGCGTAACGTTGCCCCGGCGTACATGGACCGG
GTGTATGAGTATCTCACAGAGCGCCGTACAAAGTCACCTACGCACATCCTGCGT
ACGCTCCGTGCCAGTGCCGAGAACGTGCACTACGGGCACGAGCCTTGGACCAA
TGCCCAGAACATCTCCGTAGGGCGTCTGCTGTGTGCCGCAGTATTCGAGACGGG
TCTGTTCCAGTGGAAGACGGGCAGCGGGAACCTGAGCATGCTCTACCCCGCAG
ACGACGTTATGGAAGCATTTCAGCAGCTAGTGGAATCTGCTGACACTGTGACG
ATGAAGCCTCCTATGCTGGTGCCGCCGGTGCAGCACACTACGATGTGGGACGG
CGGGTACCTCACCCCAATCGATAACCGCGGTACGTACCATAACTCGCACATCG
ACCGCACCAGACTGCGTGAAGTAGCGGAAGCATTTAAGTCTGCGGACGGCATC
AAGAAGGCGCTTAATAAGGCACAGGAAACTCCGTACCGTATTAATAAGCGCAT
ACTGGAACTGGTGCAAGAAGCACGGGCCCTAGGTATCGGAGTGGGTATGCCCC
GCTCAGTGCCGGAGCCTAAACCGGAGTGGTACTTGGATGGCGTACCAAAAGAG
AACTACACAGAGGAAGAACTGGACCGCTTCGGTGAGTGGAAGACGCGTATGTC
TCTGTGGTACAGTGCTGACCGTAAGCGTGTGTCGCAACTGCGCAGCCTTCTGAC
TACGTTAGAAATGGCAGAGGAATTCAAAGATGAGAAAGCTCTTTACTTCCCGA
CTTGTGTGGACTGGCGCTACCGCCTGTACTTCAAGTCCTCACTGCATCCCCAAG
GTTCTGATTTGCAGAAAGCCCTTCTTGAGTTTGGCAGAGGAAAACCTCTGGGTG
ATCGCGGCTTATTCTGGCTCAAGGTGCATGTCGCCACATGCTTTGGTTATGACA
AGACCTTATTCGAAGACCGCGCAGCTTGGGTTGATGCGAACTTTGCAGAGATTG
AGCAGCTTACAGTTTCACCGTTTGATTGCCCTGCTTTCACCTCCGCGGACAGCC
CTTGGTGCTTATTGGCCGCCGCTATCGACCTGGTTAATGCTGTGCGTTCTGGAT
GCCCAGAAGAGCATATTAGCCGAATCCCAGTTGCTATGGACGCTACAAACTCT
GGTGGACAGCACCTCTCAGCGCTCCTGAGAGACCCTGTAGGCGGTCGTCTGAC
GAACCTGTACTGGGAAGGTAACGACAAGAAAGCGGACCTGTACATGGATGTGA
AGCGCCGTACGGACGAGAAGGTGATACTGGACCTGGACAAGGAGGACTTCATT
ATCCAGAGCACGTACTGGAGAGAGAACGAAATCACCCGCAGCATGACCAAGC
GCCCCAGTATGACCTACTTCTACAGCGCCACGGTGCGTAGCTGCAGCGACTACA
TCTTTGAAGGCGCTTGCGCTGAGGGATACGAGGGTACCGATACTAACAGTCTGT
GGAACCTGTCGTGTTACCTGGCACCGCGTATGCGTGCCGCTATCGAGGAGGCA
AACCCCGCTGCTGCGGCAGTTATGGGGTACTTGCAGAACCTAGCTAGACGTGT
ACCGGCAAGCCAGCACCTGCAGTGGTATACGCCGCTGGGTGGGCTCGTAATGA
ACCGCTACACGCAGCGTGAAGAAGTGCGCGTACGTATTGACTGCATGAACCTA
TCGGCGGTACTGGTACACAACCGGGACTTCAAGACCTGCAACAAGCGTAAGGC
AGCCTCCGGGATTGCCCCGAACTTTGTGCATAGTCTGGACAGTACGCACTTGAT
GATGGTGCTCTGCGCTGCGGAGGGTCTGGATATTGTGCCGATTCACGACTCACT
GGCTACTCACGCAGCCGACGTTGATGATATGCACCGACACATCCGCGAACAGT
TTGTGCGCCTCTACGAGGAGAATGACCTGCTTGGCGACATTACTCGCGCGGCGG
CAGCAGCCGGGGCAGACTTGACGGACCTGGACATGCCTGAGGTGGGTACTTTG
GACATCCGTCAAGTGCTAGAATCCCCGTTCTTCTTCTGC.

### Expression and Purification

In some embodiments, a Klebsiella phage KP34 RNA polymerase that is suitable for use with the methods for manufacturing mRNA described herein can be recombinantly expressed in a bacterial cell, such as Escherichia coli.

In some embodiments, the Klebsiella phage KP34 RNA polymerase is purified from a crude bacterial extract, e.g., by affinity purification. For example, the recombinant protein may be fused to an affinity tag (e.g., a His tag) for ease of purification. The affinity-purified enzyme can be used directly for IVT. The presence of a tag does not typically interfere with the polymerase activity. For instance, as shown in the examples, a tag added to the N-terminus of the Klebsiella phage KP34 RNA polymerase does not interfere with the polymerase activity.

In other embodiments, hydrophobic interaction chromatography (HIC) is used to purify KP34. For example, a HIC column comprising a butyl ligand such as Capto™ butyl can be used to purify KP34. Purification by HIC avoids addition of an affinity tag to purify the recombinant protein.

The inventors found that overexpression of Klebsiella phage KP34 RNA polymerase in bacterial cells can result in aggregation. The aggregates were found to be enzymatically inactive. Therefore, in some embodiments, an isolated Klebsiella phage KP34 RNA polymerase may be further purified to remove enzymatically inactive aggregates. For example, enzymatically inactive aggregates may be removed by gel filtration (e.g., of an affinity-purified preparation).

In particular embodiments, recombinantly expressed Klebsiella phage KP34 RNA polymerase is isolated from a bacterial extract (e.g., using affinity purification), which is followed by chromatography, such as size exclusion chromatography (e.g., gel purification) to remove enzymatically inactive aggregates. In some embodiments, a gel filtration column (e.g., Superdex™ 200) is used to remove enzymatically inactive aggregates.

In some embodiments, a Klebsiella phage KP34 RNA polymerase for use with a method of the invention comprises less than 5% enzymatically inactive aggregates (e.g., less than 4%, 3%, 2%, or 1%). In some embodiments, the Klebsiella phage KP34 RNA polymerase comprises less than 1% enzymatically inactive aggregates. In some embodiments, the Klebsiella phage KP34 RNA polymerase is substantially free of enzymatically inactive aggregates. The presence of aggregates can be determined, e.g., by gel filtration or Western blot.

In some embodiments, at least 95% (e.g., at least 96%, 97%, or 98%) of a Klebsiella phage KP34 RNA polymerase for use with a method of the invention is in monomeric form. In some embodiments, 99% of the Klebsiella phage KP34 RNA polymerase is in monomeric form.

In some embodiments, a Klebsiella phage KP34 RNA polymerase for use with a method of the invention is provided as a fusion protein comprising a solubility tag, e.g., to improve recombinant expression in E. coli. In typical embodiments, the solubility tag is added to the N-terminus of the Klebsiella phage KP34 RNA polymerase. As shown herein, suitable solubility tags include InfB7, DxsN, P17, SmbP, and T7A3.

### DNA Template

Klebsiella phage KP34 RNA polymerase is a DNA-dependent RNA polymerase. DNA-dependent RNA polymerase initiates transcription by contacting a suitable promoter sequence in a DNA template. Accordingly, a typical DNA template for use with the methods of the invention comprises a Klebsiella phage KP34 promoter sequence, for example a promoter, operably linked to a nucleic acid sequence encoding an mRNA transcript.

### Promoter

Any promoter that can be recognized by a Klebsiella phage KP34 RNA polymerase may be used in the present invention. An exemplary Klebsiella phage KP34 core promoter comprises the following sequence: 5′-TAATGTTACAGGAGTA-3′ (SEQ ID NO: 4). Alternatively, an exemplary Klebsiella phage KP34 core promoter comprises the following sequence: 5′-ATGTTACAGGAGTA-3′ (SEQ ID NO: 5).

A suitable KP34 promoter for the present invention may be at least about 70%, 75%, 80%, 85%, 90%, 95%, or 99% (e.g., about 70%, 75%, 80%, 85%, 90%, 95%, or 99%) identical to SEQ ID NO: 4. Alternatively, a suitable KP34 promoter may be at least about 70%, 75%, 80%, 85%, 90%, 95%, or 99% (e.g., about 70%, 75%, 80%, 85%, 90%, 95%, or 99%) identical to SEQ ID NO: 5. In some embodiments, a promoter homologous to SEQ ID NO: 4 or 5 may be used in implementing the invention. In some embodiments, a KP34 promoter suitable in the present invention may include one or more additional nucleotides 5′ and/or 3′ to any one of the promoter sequences described herein.

In some embodiments, the nucleic acid sequence 3′ adjacent to the core promoter sequence is optimized to improve mRNA yield during in vitro synthesis. For example, the KP34 promoter may comprise three guanines 3′ adjacent to a core promoter sequence of SEQ ID NO: 4 or 5. An exemplary Klebsiella phage KP34 promoter thus comprises the following sequence: TAATGTTACAGGAGTAGGG (SEQ ID NO: 6). The inventors have observed particularly good performance with this promoter sequence when the 3′ adjacent sequence to SEQ ID NO: 6 is A or GA.

In some embodiments, the additional nucleotides 3′ adjacent to a KP34 core promoter (e.g., SEQ ID NO: 4 or 5) are GGA, GGGA, or GGGGA. Thus, an exemplary KP34 promoter may comprise one of the following nucleic acid sequences, in which the minimal core promoter sequence (SEQ ID NO: 5) is shown in bold:

(SEQ ID NO: 7)
TAATGTTACAGGAGTAGGGGA;
(SEQ ID NO: 8)
TAATGTTACAGGAGTAGGGA;
or
(SEQ ID NO: 9)
TAATGTTACAGGAGTAGGA.

The inventors observed that the nucleic acid sequence further downstream of the core promoter sequence can also impact performance. Accordingly, in some embodiments, the additional nucleotides 3′ adjacent to a KP34 core promoter (e.g., SEQ ID NOs: 4 or 5) are GₙAN₁N₂N₃N₄W, wherein n is 2, 3 or 4, each of N₁₋₄ is any one of A, C, G or T, and W is A or T. In some embodiments, the additional nucleotides 3′ adjacent to a KP34 core promoter (e.g., SEQ ID NOs: 4 or 5) are GₙAN₁N₂N₃N₄N₅WV, wherein n is 2, 3 or 4, each of N₁₋₅ is any one of A, C, G or T, W is A or T, and V is C or T. In some embodiments, N₁₋₅ are independently selected from C, A and G, and W is A or T. In some embodiments, N₁ is C or G, N₂ is A, N₃ is A, C or G and N₄ and N₅ are independently selected from A and G. Exemplary nucleic acid sequences 3′ adjacent to the core promoter can be GGGGACAAGATC (SEQ ID NO: 37), GGAGACAGATC (SEQ ID NO: 38), GGAGAGAGATC (SEQ ID NO: 39), or GGAGACAGTTT (SEQ ID NO: 40). Typically, the nucleic acid sequence 3′ adjacent to the core promoter is GGGGACAAGATC (SEQ ID NO: 37) or GGAGACAGTTT (SEQ ID NO: 40), e.g., SEQ ID NO: 40. Accordingly, in some embodiments, KP34 promoter comprises the following nucleic acid sequence, in which the minimal core promoter sequence (SEQ ID NO: 5) is shown in bold:

(SEQ ID NO: 41)
TAATGTTACAGGAGTAGGGGACAAGATC.

Similarly, the sequence provided in SEQ ID NOs: 38, 39, or 40 may be combined with a KP34 promoter sequence such as that set forth in SEQ ID NO: 5, as set forth, e.g., in SEQ ID NOs: 42-44. Additional exemplary sequences are set forth in SEQ ID NOs: 45-48.

In some embodiments, KP34 promoter may comprise one of the following nucleic acid sequences, in which the minimal core promoter sequence (SEQ ID NO: 5) is shown in bold: TAATGTTACAGGAGTAGGₙAN₁N₂N₃N₄W (SEQ ID NO: 10) wherein n is 1-5 i.e., G, GG, GGG, GGGG, or GGGGG. In particular embodiments, n is 1 or 3 (i.e., G or GGG). In particular embodiments, N₁N₂N₃N₄ is CAGA. Accordingly, exemplary KP34 promoter sequences comprise TAATGTTACAGGAGTAGGGGGACAGAT (SEQ ID NO: 11); or TAATGTTACAGGAGTAGGGGGACAGAA of SEQ ID NO: 12). A further exemplary promoter sequence comprises TAATGTTACAGGAGTAGGACAGATC (SEQ ID NO: 36). Further exemplary promoter sequences comprise TAATGTTACAGGAGTAGGACAGATC (SEQ ID NO: 36), TAATGTTACAGGAGTAGGGGACAAGATC (SEQ ID NO: 41), or TAATGTTACAGGAGTAGGAGACAGTTT (SEQ ID NO: 44).

### 5′ and 3′ Untranslated Regions

A nucleotide sequence encoding an mRNA transcript for expression of a polypeptide or protein usually comprises a 5′ untranslated region (5′ UTR), a coding region for a polypeptide of interest, and a 3′ untranslated region (3′ UTR).

In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

In some embodiments, the nucleotide sequence comprises a 5′ UTR different from the 5′ UTR present in a naturally occurring mRNA encoding the polypeptide of interest.

In some embodiments, the nucleotide sequence comprises a 3′ UTR different from the 3′ UTR present in a naturally occurring mRNA encoding the polypeptide of interest.

For example, suitable 5′ and 3′ UTRs are described in WO2012/075040, which is incorporated herein by reference.

In certain embodiments, the 5′ and/or 3′ UTR sequences can be derived from mRNA which are stable (e.g., globin, actin, GAPDH, tubulin, histone, or citric acid cycle enzymes) to increase the stability of the mRNA. For example, a 5′ UTR sequence may include a partial sequence of a CMV immediate-early 1 (IE1) gene, or a fragment thereof, to improve the nuclease resistance and/or improve the half-life of the mRNA. Also contemplated is the inclusion of a sequence encoding human growth hormone (hGH), or a fragment thereof, to the 3′ end or untranslated region of the mRNA. Exemplary 5′ UTRs include a sequence derived from a CMV immediate-early 1 (IE1) gene (U.S. Publication Nos. 2014/0206753 and 2015/0157565, each of which is incorporated herein by reference), or the sequences provided in Example 1 of U.S. Publication No. 2016/0151409, incorporated herein by reference.

In various embodiments, the 5′ UTR may be derived from the 5′ UTR of a TOP gene. TOP genes are typically characterized by the presence of a 5′-terminal oligopyrimidine (TOP) tract. Furthermore, most TOP genes are characterized by growth-associated translational regulation. However, TOP genes with a tissue specific translational regulation are also known. In certain embodiments, the 5′ UTR derived from the 5′ UTR of a TOP gene lacks the 5′ TOP motif (the oligopyrimidine tract) (e.g., U.S. Publication Nos. 2017/0029847, 2016/0304883, 2016/0235864, and 2016/0166710, each of which is incorporated herein by reference).

In certain embodiments, the 5′ UTR is derived from a ribosomal protein Large 32 (L32) gene (U.S. Publication No. 2017/0029847, supra).

In certain embodiments, the 5′ UTR is derived from the 5′ UTR of a hydroxysteroid (17-b) dehydrogenase 4 gene (HSD17B4) (U.S. Publication No. 2016/0166710, supra).

In certain embodiments, the 5′ UTR is derived from the 5′ UTR of an ATP5A1 gene (U.S. Publication No. 2016/0166710, supra).

In some embodiments, an internal ribosome entry site (IRES) is used instead of a 5′ UTR.

A 5′ UTR for use with the invention may comprise one of the nucleic acid sequences shown in Table 1. The nucleotides immediately 3′ adjacent to the KP34 core promoter are shown in bold.

TABLE 1
Nucleic acid sequences comprised in the 5′ UTR

| SEQ ID NO | Sequence |
|---|---|
| 13 | GGGGGACAGATCGCCTGGAGACGC |
| 14 | GGGGACAGATCGCCTGGAGACGC |
| 15 | GGGACAGATCGCCTGGAGACGC |
| 16 | GGGCAGATCGCCTGGAGACGC |
| 17 | GGACAGATCGCCTGGAGACGC |
| 18 | GGGGGACAGAACGCCTGGAGACGC |

For example, the sequences provided in Table 1 may be combined with a KP34 promoter to form the following sequences, with the minimal core promoter sequence (SEQ ID NO: 5) shown in bold:

(SEQ ID NO: 19)
TAATGTTACAGGAGTAGGGGACAGATCGCCTGGAGACGC.
(SEQ ID NO: 20)
TAATGTTACAGGAGTAGGGACAGATCGCCTGGAGACGC.

An exemplary 5′ UTR for use with the invention has the following sequence: CAGATCGCCTGGAGACGCCATCCACGCTGTTTTGACCTCCATAGAAGACACCG GGACCGATCCAGCCTCCGCGGCCGGGAACGGTGCATTGGAACGCGGATTCCCC GTGCCAAGAGTGACTCACCGTCCTTGACACG (SEQ ID NO: 21). The underlined 5′ nucleic acid sequence is present in the sequences of Table 1 (except for SEQ ID NO: 18, which includes a further modified sequence). For example, the underlined 5′ nucleic acid sequence of SEQ ID NO: 21 is present in the KP34 promoter sequence of SEQ ID NO: 33 in Table 10 (SEQ ID NOs: 32, 34, and 35 include a further modified sequence).

### Template Linearization

The in vitro transcribed mRNA is typically transcribed from a DNA template which is linearized using a restriction enzyme. In this context, any restriction enzyme (see, e.g., Roberts et al. (2015) Nucl. Acids Res. 43; D1: D298-D299) may be used. Generally, the restriction enzyme is a type II restriction enzyme, such as a type IIP or type IIS restriction enzyme. In some embodiments, the restriction enzyme is EcoRI, BciVI, Spel, Xbal, Ndel, Aflll, Sacl, Kpnl, Smal, BamHI, Sail, Sbfl, Pstl, BspQI, or Hindlll.

In some embodiments, the linearized DNA template has blunt ends.

### Sequence Optimization

In some embodiments, the DNA sequence to be transcribed may be optimized to facilitate more efficient transcription and/or translation. For example, the DNA sequence may be optimized regarding cis-regulatory elements (e.g., TATA box, termination signals, and protein binding sites), artificial recombination sites, chi sites, CpG dinucleotide content, negative CpG islands, GC content, polymerase slippage sites, and/or other elements relevant to transcription; the DNA sequence may be optimized regarding cryptic splice sites, mRNA secondary structure, stable free energy of mRNA, repetitive sequences, RNA instability motif, and/or other elements relevant to mRNA processing and stability; the DNA sequence may be optimized regarding codon usage bias, codon adaptability, internal chi sites, ribosomal binding sites (e.g., IRES), premature polyA sites, Shine-Dalgarno (SD) sequences, and/or other elements relevant to translation; and/or the DNA sequence may be optimized regarding codon context, codon-anticodon interaction, translational pause sites, and/or other elements relevant to protein folding. A suitable sequence optimization method is described in International Patent Publication No. WO 2021/226461A1, which is incorporated herein by reference.

### In Vitro Transcription (IVT)

Various methods for synthesizing mRNA via IVT are described in US Patent Publication No. US 2018/0258423 and International Patent Publication No. WO 2021/168052A1, which are incorporated herein by reference, and can be used to practice the present invention. Briefly, IVT is typically performed with a reaction mixture comprising a DNA template, a pool of ribonucleotide triphosphates, a buffering reagent (that may include DTT), and one or more salts (e.g., MgCl₂ and NaCl). A typical IVT reaction buffer may also include spermidine. The exact conditions will vary according to the specific application.

### Template Concentration

The concentration of the DNA template in an IVT reaction ranges from 0.05 mg/mL to 0.5 mg/mL. In some embodiments, the concentration of the DNA template is 0.05 mg/mL, 0.06 mg/mL, 0.07 mg/mL, 0.08 mg/mL, 0.09 mg/mL, 0.1 mg/mL, 0.11 mg/mL, 0.12 mg/mL, 0.13 mg/mL, 0.14 mg/mL, 0.15 mg/mL, 0.16 mg/mL, 0.17 mg/mL, 0.18 mg/mL, 0.19 mg/mL, 0.2 mg/mL, 0.21 mg/mL, 0.22 mg/mL, 0.23 mg/mL, 0.24 mg/mL, 0.25 mg/mL, 0.26 mg/mL, 0.27 mg/mL, 0.28 mg/mL, 0.29 mg/mL, 0.3 mg/mL, 0.31 mg/mL, 0.32 mg/mL, 0.33 mg/mL, 0.34 mg/mL, 0.35 mg/mL, 0.36 mg/mL, 0.37 mg/mL, 0.38 mg/mL, 0.39 mg/mL, 0.4 mg/mL, 0.41 mg/mL, 0.42 mg/mL, 0.43 mg/mL, 0.44 mg/mL, 0.45 mg/mL, 0.46 mg/mL, 0.47 mg/mL, 0.48 mg/mL, 0.49 mg/mL, or 0.5 mg/mL.

### Polymerase Concentration

The concentration of the Klebsiella phage KP34 RNA polymerase in an IVT reaction ranges from 0.01 to 0.5 mg/mL. In some embodiments, the concentration of the Klebsiella phage KP34 RNA polymerase is 0.01 mg/mL, 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.11 mg/mL, 0.12 mg/mL, 0.13 mg/mL, 0.14 mg/mL, 0.15 mg/mL, 0.16 mg/mL, 0.17 mg/mL, 0.18 mg/mL, 0.19 mg/mL, 0.2 mg/mL, 0.21 mg/mL, 0.22 mg/mL, 0.23 mg/mL, 0.24 mg/mL, 0.25 mg/mL, 0.26 mg/mL, 0.27 mg/mL, 0.28 mg/mL, 0.29 mg/mL, 0.3 mg/mL, 0.31 mg/mL, 0.32 mg/mL, 0.33 mg/mL, 0.34 mg/mL, 0.35 mg/mL, 0.36 mg/mL, 0.37 mg/mL, 0.38 mg/mL, 0.39 mg/mL, 0.4 mg/mL, 0.41 mg/mL, 0.42 mg/mL, 0.43 mg/mL, 0.44 mg/mL, 0.45 mg/mL, 0.46 mg/mL, 0.47 mg/mL, 0.48 mg/mL, 0.49 mg/mL, or 0.5 mg/mL.

### Buffering Reagent

IVT typically takes place in the presence of a buffering agent. In some embodiments, the buffering agent is selected from Tris, HEPES, ammonium sulfate, sodium bicarbonate, sodium citrate, sodium acetate, potassium phosphate, and sodium phosphate. In one embodiment, the buffering agent is Tris-HCl. In a further embodiment, Tris-HCl is present at a concentration of less than 40 mM. In some embodiments, Tris-HCl is present at a concentration of about 25 mM.

pH

The pH of the reaction mixture may be between about 6 to 8.5, from 6.5 to 8.0, from 7.0 to 7.5, and in some embodiments, the pH is 7.5.

### Temperature

In some embodiments, IVT takes place at a temperature from about 37° C. to about 42° C. In some embodiments, IVT takes place at a temperature of about 37° C., 38° C., 39° C., 40° C., 41° C., or 42° C.

### Co-Factor

Most polymerases include a divalent cation as a co-factor. Accordingly, in some embodiments, IVT takes place in the presence of divalent cation, e.g., Mn²⁺ or Mg²⁺.

In particular embodiments, IVT takes place in the presence of magnesium chloride (MgCl₂). The inventors found that Klebsiella phage KP34 RNA polymerase has high activity in the presence of MgCl₂ concentrations exceeding 20 mM. Accordingly, in some embodiments, the MgCl₂ concentration is greater than 20 mM. In some embodiments, the MgCl₂ concentration is greater than 20 mM and less than 40 mM (e.g., less than 30 mM). In some embodiments, the MgCl₂ concentration is about 25 mM.

### Salt

To provide optimal conditions for IVT, a salt is typically included in the reaction mixture. In some embodiments, IVT takes place in the presence of sodium chloride (NaCl). In some embodiments, the NaCl concentration is less than 20 mM. In some embodiments, the NaCl concentration is between 0.05 mM and 15 mM. In some embodiments, the NaCl concentration is between 0.1 mM and 10 mM. In some embodiments, the NaCl concentration is between 0.1 mM and 5 mM. In some embodiments, the NaCl concentration is between 0.1 mM and 2 mM. In some embodiments, the NaCl concentration is between 0.1 mM and 1 mM. In some embodiments, the NaCl concentration is about 0.5 mM.

### Ribonucleotides

In some embodiments, the concentration of each ribonucleotide (e.g., ATP, UTP, GTP, and CTP) in a reaction mixture is between about 0.1 mM and about 10 mM, e.g., between about 1 mM and about 10 mM, between about 2 mM and about 10 mM, between about 3 mM and about 10 mM, between about 1 mM and about 8 mM, between about 1 mM and about 6 mM, between about 3 mM and about 10 mM, between about 3 mM and about 8 mM, between about 3 mM and about 6 mM, between about 4 mM and about 7 mM, between about 4 mM and about 6 mM, or between about 4 mM and about 5 mM. In some embodiments, each ribonucleotide is at about 5 mM in a reaction mixture.

In some embodiments, the total concentration of ribonucleotide (for example, ATP, GTP, CTP, and UTPs combined) used in the reaction is between about 1 mM and about 40 mM. In some embodiments, the total concentration of ribonucleotide (for example, ATP, GTP, CTP, and UTPs combined) used in the reaction is between about 1 mM and about 30 mM, between about 1 mM and about 28 mM, between about 1 mM and about 25 mM, or between about 1 mM and about 20 mM.

In some embodiments, the total ribonucleotide concentration is less than about 30 mM. In some embodiments, the total ribonucleotide concentration is less than about 25 mM. In some embodiments, the total ribonucleotide concentration is less than about 20 mM. In some embodiments, the total ribonucleotide concentration is less than about 15 mM. In some embodiments, the total ribonucleotide concentration is less than about 10 mM.

### Modified RNA

In some embodiments, mRNA transcripts are synthesized with one or more modifications (i.e., as modified mRNA), wherein the modification refers to chemical or biological modifications comprising backbone modifications, sugar modifications, or base modifications. A backbone modification is a modification in which phosphates of the backbone of the nucleotides of the RNA are chemically modified (e.g., phosphorothioates and 5′-N-phosphoramidite linkages). A sugar modification is a chemical modification of the sugar of the nucleotides of the RNA (e.g., 2′-fluororibose, ribose, 2′-deoxyribose, arabinose, and hexose). A base modification is a chemical modification of the base moiety of the nucleotides of the RNA.

In a particular embodiment, modified mRNA comprises a modified ribonucleotide, such as ribonucleotide analogue (e.g., adenosine analogue, guanosine analogue, cytidine analogue, and/or uridine analogue). The presence of a modified ribonucleotide may render the mRNA more stable and/or less immunogenic than a control mRNA with the same sequence but containing only naturally occurring ribonucleotides.

The modified ribonucleotide typically takes the place of a naturally occurring nucleotide. Accordingly, in one aspect, the in vitro transcribed mRNA of the invention comprises both unmodified and modified ribonucleotides. Such in vitro transcribed mRNA can be prepared by including a modified ribonucleoside in the IVT reaction mixture, typically in place of a naturally occurring ribonucleoside (e.g., N1-methylpseudouridine in place of uridine). This results in in vitro transcribed mRNA in which 100% of the naturally occurring ribonucleotide is replaced by a corresponding modified ribonucleotide (e.g., 100% of the uridines are replaced with N1-methyl-pseudouridine). In some embodiments, only a portion of the naturally occurring ribonucleoside (e.g., at least 1%, 5%, 10%, 15%, 20% or 25% of the naturally occurring ribonucleoside) is replaced with a modified ribonucleoside. In some embodiments, one or more naturally occurring ribonucleosides is replaced with a modified ribonucleoside. For example, two or more ribonucleosides may be modified ribonucleosides (e.g., uridines may be replaced with 2-thio-uridine and cytidines may be replaced with 5-methylcytidine). For example, 25% of the uridines may be replaced with 2-thio-uridine and/or 25% of cytidine residues may be replaced with 5-methylcytidine.

In some embodiments, the modified ribonucleoside comprises at least one modification selected from a modified sugar, and a modified nucleobase relative to the corresponding naturally occurring ribonucleoside.

The modified ribonucleoside can be a modified uridine, cytidine, adenosine, or guanosine. Some exemplary chemical modifications of ribonucleosides in the mRNA molecule include, e.g., pyridine-4-one ribonucleoside, 5-aza-uridine, 2-thio-5-aza-uridine, 2-thiouridine, 4-thio pseudouridine, 2-thio-pseudouridine, 5-hydroxyuridine, 3-methyluridine, 5-carboxymethyl uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N⁶-glycinylcarbamoyladenosine, N⁶-threonylcarbamoyladenosine, 2-methylthio-N⁶-threonyl carbamoyladenosine, N⁶,N⁶-dimethyladenosine, 7-methyladenine, 2-methylthioadenine, 2-methoxyadenine, inosine, 1-methyl-inosine, wyosine, wybutosine, 7-deaza guanosine, 7-deaza-8-aza-guanosine, 6-thio guanosine, 6-thio-7-deazaguanosine, 6-thio-7-deaza-8-aza-guanosine, 7-methyl-guanosine, 6-thio-7-methylguanosine, 7-methylinosine, 6-methoxy guanosine, 1-methylguanosine, N²-methylguanosine, N²,N²-dimethylguanosine, 8-oxo-guanosine, 7-methyl-8-oxo-guanosine, 1-methyl-6-thio-guanosine, N²-methyl-6-thio-guanosine, and N²,N²-dimethyl-6-thio-guanosine.

In some embodiments, the modified ribonucleoside is a modified uridine selected from pseudouridine, pyridine-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy uridine, 5-aminoallyl-uridine, 5-halo-uridine (e.g., 5-iodom uridine or 5-bromo uridine), 3-methyl uridine, 5-methoxy-uridine, uridine-5-oxyacetic acid, uridine-5-oxyacetic acid methyl ester, 5-carboxymethyl-uridine, 1-carboxymethyl-pseudouridine, 5-carboxyhydroxymethyl uridine, 5-carboxyhydroxymethyl-uridine methyl ester, 5-methoxycarbonylmethyluridine, 5-methoxycarbonylmethyl-2-thio-uridine, 5-aminomethyl-2-thiouridine, 5-methylaminomethyl uridine, 5-methylaminomethyl-2-thio-uridine, 5-methylaminomethyl-2-selenouridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl pseudouridine, 5-taurinomethyl-2-thio-uridine, 1-taurinomethyl-4-thio-pseudouridine, 5-methyl-uridine (m⁵U, e.g., having the nucleobase deoxythymine), 1-methyl-pseudouridine, 5-methyl-2-thio-uridine, 1-methyl-4-thio-pseudouridine, 4-thio-1-methyl-pseudouridine, 3-methyl-pseudouridine), 2-thio-1-methyl-pseudouridine, 1-methyl-1-deaza-pseudouridine, 2-thio-1-methyl-1-deaza-pseudouridine, dihydrouridine, dihydropseudouridine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N¹-methyl-pseudouridine, 3-(3-amino-3-carboxypropyl) uridine, 1-methyl-3-(3-amino-3-carboxypropyl) pseudouridine, 5-(isopentenylaminomethyl) uridine, 5-(isopentenylaminomethyl)-2-thio-uridine, alpha-thio-uridine, 2′-O-methyl uridine, 5,2′-O-dimethyl uridine, 2′-O-methyl-pseudouridine, 2-thio-2′-O-methyl uridine, 5-methoxycarbonylmethyl-2′-O-methyl uridine, 5-carbamoylmethyl-2′-O-methyl uridine, 5-carboxymethylaminomethyl-2′-O-methyl uridine, 3,2′-O-dimethyl uridine, 5-(isopentenylaminomethyl)-2′-O-methyl uridine, 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) uridine.

In some embodiments, the modified uridine is selected from N¹-methylpseudouridine, pseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In some embodiments, the modified uridine is N¹-methylpseudouridine.

In some embodiments, the modified ribonucleoside is a modified cytidine selected from 5-aza-cytidine, 6-aza-cytidine, pseudoisocytidine, 3-methylcytidine, N⁴-acetyl cytidine, 5-formyl-cytidine, N⁴-methylcytidine, 5-methylcytidine, 5-halo cytidine (e.g., 5-iodo cytidine), 5-hydroxy methylcytidine, 1-methyl-pseudoisocytidine, pyrrolo-cytidine, pyrrolo-pseudoisocytidine, 2-thio-cytidine, 2-thio-5-methylcytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio zebularine, 2-thio-zebularine, 2-methoxy cytidine, 2-methoxy-5-methylcytidine, 4-methoxy pseudoisocytidine, 4-methoxy-1-methyl-pseudoisocytidine, lysidine, alpha-thio-cytidine, 2′-O-methylcytidine, 5,2′-O-dimethyl cytidine, N⁴-acetyl-2′-O-methylcytidine, N⁴,2′-O-dimethyl cytidine, 5-formyl-2′-O-methylcytidine, N⁴,N⁴,2′-O-trimethyl cytidine, 1-thio-cytidine, 2′-F-ara-cytidine, 2′-F cytidine, and 2′-OH-ara-cytidine.

In some embodiments, the modified ribonucleoside is a modified pyrimidine ribonucleoside. In some embodiments, the modified ribonucleoside is selected from pseudouridine, N¹-methylpseudouridine, 5-methylcytidine, 5-methoxyuridine, and any combination thereof. In some embodiments, both cytidine and uridine are replaced with modified nucleosides (e.g., N¹-methylpseudouridine and 5-methylcytidine).

In some embodiments, the modified ribonucleoside is a modified purine ribonucleoside. In some embodiments, the modified ribonucleoside is a modified adenosine selected from 2-amino purine, 2,6-diamino purine, 2-amino-6-halo purine (e.g., 2-amino-6-chloro purine), 6-halo purine (e.g., 6-chloro purine), 2-amino-6-methyl purine, 8-azido adenosine, 7-deaza-adenine, 7-deaza-8-aza adenine, 7-deaza-2-amino purine, 7-deaza-8-aza-2-amino purine, 7-deaza-2,6-diamino purine, 7-deaza-8-aza-2,6-diamino purine, 1-methyladenosine, 2-methyl adenine, N⁶-methyladenosine, 2-methylthio-N⁶-methyladenosine, N⁶-isopentenyl adenosine, 2-methylthio-N⁶-isopentenyl adenosine, N⁶-(cis-hydroxyisopentenyl) adenosine, 2-methylthio-N⁶-(cis-hydroxyisopentenyl) adenosine, N⁶-glycinylcarbamoyl adenosine, N⁶-threonylcarbamoyl adenosine, N⁶-methyl-N⁶-threonylcarbamoyl adenosine, 2-methylthio-N⁶-threonylcarbamoyl adenosine, N⁶,N⁶-dimethyl adenosine, N⁶-hydroxynorvalylcarbamoyl adenosine, 2-methylthio-N⁶-hydroxynorvalylcarbamoyl adenosine, N⁶-acetyl adenosine, 7-methyladenine, 2-methylthio-adenine, 2-methoxyadenine, alpha-thio-adenosine, 2′-O-methyladenosine, N⁶,2′-O-dimethyl adenosine, N⁶,N⁶,2′-O-trimethyl adenosine, 1,2′-O-dimethyl adenosine, 2′-O-ribosyl adenosine (phosphate), 2-amino-N⁶-methyl purine, 1-thio-adenosine, 8-azido-adenosine, 2′-F-ara-adenosine, 2′-F adenosine, 2′-OH-ara-adenosine, and N⁶-(19-amino-pentaoxanonadecyl) adenosine.

In some embodiments, the modified ribonucleoside is a modified guanosine selected from inosine, 1-methyl inosine, wyosine, methylwyosine, 4-demethyl wyosine, isowyosine, wybutosine, peroxywybutosine, hydroxywybutosine, undermodified hydroxywybutosine, 7-deaza-guanosine, queuosine, epoxyqueuosine, galactosyl queuosine, mannosyl queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methylguanosine, 6-thio-7-methylguanosine, 7-methyl inosine, 6-methoxy guanosine, 1-methylguanosine, N²-methyl-guanosine, N²,N²-dimethyl guanosine, N^{2,7}-dimethyl guanosine, N²,N^{2,7}-dimethyl guanosine, 8-oxo-guanosine, 7-methyl-8-oxo-guanosine, 1-methylguanosine, N²-methyl-6-thio-guanosine, N²,N²-dimethyl-6-thio-guanosine, alpha-thio-guanosine, 2′-O-methylguanosine, N²-methyl-2′-O-methylguanosine, N²,N²-dimethyl-2′-O-methylguanosine, 1-methyl-2′-O-methylguanosine, N^{2,7}-dimethyl-2′-O-methylguanosine, 2′-O-methyl inosine, 1,2′-O-dimethyl inosine, 2′-O-ribosyl guanosine (phosphate), 1-thio-guanosine, 06-methylguanosine, 2′-F-ara guanosine, and 2′-F guanosine.

In some embodiments, the modified ribonucleoside is a ribonucleoside analogue selected from 2-aminoadenosine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, pseudouridine (e.g., N¹-methylpseudouridine), 2-thiouridine, and 2-thiocytidine. See, e.g., U.S. Pat. No. 8,278,036 or WO 2011/012316 for a discussion of 5-methylcytidine, pseudouridine, and 2-thio-uridine and their incorporation into mRNA.

In some embodiments, the modified ribonucleoside is selected from pseudouridine, N¹-methylpseudouridine, 2-thiouridine, 4′-thiouridine, 5-methylcytidine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, and 2′-O-methyl uridine.

In some embodiments, the in vitro transcribed mRNA may be RNA wherein 25% of uracil residues are 2-thio-uracil and 25% of cytosine residues are 5-methylcytosine. Teachings for the use of such modified RNA are disclosed in US Patent Publication US 2012/0195936 and international publication WO 2011/012316, both of which are hereby incorporated by reference in their entirety. In some embodiments, the in vitro transcribed mRNA may be RNA where 100% of uracil residues are N¹-methylpseudouracil (also occasionally referred to as 1-methylpseudouracil).

### Time

The length of an IVT reaction may depend on the length of the mRNA transcript. In a typical embodiment, the mRNA transcript comprises at least 500 ribonucleotides. In some embodiments, the mRNA transcript comprises about 500 to about 20,000 ribonucleotides. In some embodiments, the mRNA transcript comprises about 700 to about 15,000 ribonucleotides. In some embodiments, the mRNA transcript comprises about 800 to about 12,000 ribonucleotides. In some embodiments, the mRNA transcript comprises about 1,000 to about 10,000 ribonucleotides. In some embodiments, the mRNA transcript comprises about 1,500 to about 7,000 ribonucleotides. In some embodiments, the mRNA transcript comprises about 2,000 to about 5,000 ribonucleotides.

Accordingly, the period over which IVT may take place to synthesize mRNA can vary widely. In some embodiments, IVT takes place over a period of about thirty minutes to about six hours. In some embodiments, IVT takes place over a period about sixty to about ninety minutes.

IVT can be terminated by removing the DNA template, e.g., through the addition of DNase I and a suitable buffer. For example, the polymerase reaction can be quenched by addition of DNase I and a DNase I buffer (100 mM Tris-HCl, 5 mM MgCl₂ and 25 mM CaCl₂), pH 7.6 at 10×) to facilitate digestion of the double-stranded DNA template in preparation for purification.

### Large-Scale Synthesis

In some embodiments, the mRNA is synthesized in batches. In some embodiments, the present invention relates to the large-scale manufacture of mRNA.

In some embodiments, about 1 g to about 100 kg of mRNA (e.g., 100 g to 10 kg, or 250 g to 5 kg) is synthesized in a single batch. In some embodiments, a batch comprises at least 1 g of in vitro transcribed mRNA (e.g., 5 g, 10 g, 20 g, 25 g, or 30 g). In other embodiments, a batch comprises at least 50 g of in vitro transcribed mRNA (e.g., 75 g, 100 g, 150 g, 200 g, or 250 g).

In some embodiments, a method according to the invention synthesizes at least 500 g, 750 g, 1 kg, 5 kg, 10 kg, 50 kg, 100 kg, 1000 kg, or more of mRNA in a single batch. In some embodiments, 10 kg mRNA or more is synthesized in a single batch. In some embodiments, between 10 kg and 100 kg of mRNA is synthesized in a single batch.

### Exemplary IVT Conditions

In some embodiments, a suitable reaction mixture comprises a double-stranded DNA template with a Klebsiella phage KP34 RNA polymerase-specific promoter, Klebsiella phage KP34 RNA polymerase, RNase inhibitor, pyrophosphatase, NTPs, 10 mM DTT and a reaction buffer (25 mM Tris-HCl, 2 mM spermidine, 25 mM MgCl₂, 0.5 mM NaCl, and pH 7.5). In some embodiments, this reaction mixture is incubated at 37° C. for the length of time needed to complete IVT of the mRNA transcript encoded by the DNA template.

In some embodiments, a reaction mixture includes each NTP at a concentration ranging from 1-10 mM, a DNA template at a concentration ranging from 0.01-0.5 mg/mL, and Klebsiella phage KP34 RNA polymerase at a concentration ranging from 0.01-0.1 mg/mL.

Detection of Transcriptional by-Products

During IVT, transcriptional by-products are formed in addition to the desired mRNA transcript. The present invention is based, at least in part, on the discovery that Klebsiella phage KP34 RNA polymerase produces very few transcriptional by-products e.g., dsRNA.

Various methods can be used to characterize in vitro synthesized mRNA transcripts. mRNA transcripts may be detected and quantified using any methods available in the art, e.g., using blotting (e.g., dot blot), capillary electrophoresis, chromatography, fluorescence, gel electrophoresis, HPLC, silver stain, spectroscopy, ultraviolet (UV), or UPLC, UV absorption spectroscopy with separation by capillary electrophoresis, or any combination thereof. In some embodiments, mRNA transcripts are first denatured by a glyoxal dye before analysis by gel electrophoresis (“glyoxal gel electrophoresis”).

### Non-Templated Nucleic Acids

In some embodiments, mRNA transcripts generated by IVT using Klebsiella phage KP34 RNA polymerase are substantially free of non-templated nucleic acids.

RNA-dependent 3′ end extension can be measured using an RNA-dependent RNA polymerase (RdRp) assay. A suitable RdRp assay may employ an RNA template that can anneal in an internal region of complementarity, thereby forming a stretch of double-stranded RNA in cis by looping to enable self-templated 3′end extension.

In some embodiments, about 10% by weight or less (e.g., about 5% or less, or about 2% or less) of the mRNA transcripts obtained by a method of the invention comprise non-templated nucleic acids.

Double-Stranded RNA (dsRNA)

In some embodiments, mRNA transcripts obtained by a method the invention are substantially free of dsRNA. In some embodiments, the amount of dsRNA is below the limit of detection. In some embodiments, the presence of dsRNA is determined by dot blot using antibody J2, K1, or K2 (e.g., J2). In some embodiments, the presence of dsRNA is determined by ELISA, e.g., a sandwich ELISA using antibodies J2 and K1, or antibodies K1 and K2. In some embodiments, the amount of dsRNA by weight is below the limit of detection in a 200 ng sample of in vitro synthesized mRNA. In some embodiments, the amount of dsRNA by weight is below the limit of detection in a 200 ng sample of in vitro synthesized mRNA as determined by dot blot, e.g., using monoclonal antibody J2.

In some embodiments, the mRNA transcripts comprise less than about 0.9%, 0.8%, 0.7%, 0.6%, 0.5%, 0.4%, 0.3%, 0.2% 0.1%, 0.05%, or 0.01% of dsRNA by weight, e.g., less than 0.5%. In some embodiments, the amount of dsRNA is determined by ELISA, e.g., a sandwich ELISA using antibodies J2 and K1, or antibodies K1 and K2.

In some embodiments, the amount of dsRNA generated by IVT using Klebsiella phage KP34 RNA polymerase is at least 10-fold lower relative to a corresponding method using SP6 RNA polymerase in place of the Klebsiella phage KP34 RNA polymerase. In some embodiments, the amount of dsRNA generated by IVT using Klebsiella phage KP34 RNA polymerase is at least 100-fold lower relative to a corresponding method using T7 RNA polymerase in place of the Klebsiella phage KP34 RNA polymerase.

In some embodiments, the dsRNA generated by IVT using Klebsiella phage KP34 RNA polymerase is less than about 10%, 8%, 5%, 4%, 3%, 2%, or 1% of the dsRNA generated by IVT using SP6 RNA polymerase. In particular embodiments, the dsRNA generated by IVT using Klebsiella phage KP34 RNA polymerase is less than 1% of the dsRNA generated by IVT using SP6 RNA polymerase. In some embodiments, the dsRNA generated by IVT using Klebsiella phage KP34 RNA polymerase is less than 0.5% of the dsRNA generated by IVT using SP6 RNA polymerase.

In some embodiments, the dsRNA generated by IVT using Klebsiella phage KP34 RNA polymerase is less than 10%, 8%, 5%, 4%, 3%, 2%, or 1% of the dsRNA generated by IVT using T7 RNA polymerase. In particular embodiments, the dsRNA generated by IVT using Klebsiella phage KP34 RNA polymerase is less than 1% of the dsRNA generated by IVT using T7 RNA polymerase. In some embodiments, the dsRNA generated by IVT using Klebsiella phage KP34 RNA polymerase is less than 0.5% of the dsRNA generated by IVT using T7 RNA polymerase.

### Product Integrity

The methods of the present invention yield high quality in vitro synthesized mRNA. For example, the present invention provides uniformity/homogeneity of synthesized mRNA.

In particular, a composition of the present invention includes a plurality of mRNA transcripts which are substantially full-length. For example, at least 80% of the mRNA transcripts are full-length mRNA molecules. In some embodiments, at least 90% of the mRNA transcripts are full-length mRNA molecules. In some embodiments, at least 95% of the mRNA transcripts are full-length mRNA molecules. Such a composition is said to be “enriched” for full-length mRNA molecules. In some embodiments, mRNA synthesized according to the present invention is substantially full-length.

In some embodiments, less than 20% of mRNA transcripts obtained with a method of the invention are truncated transcripts. In some embodiments, less than 10% of mRNA transcripts obtained with a method of the invention are truncated transcripts. In some embodiments, less than 5% of mRNA transcripts obtained with a method of the invention are truncated transcripts.

### Post-Synthesis Processing

A 5′ cap and/or a 3′ tail may be added after IVT. The presence of a cap is important in providing resistance to nucleases found in most eukaryotic cells. The presence of a “tail” serves to protect the mRNA from exonuclease degradation in vivo.

In some embodiments, the in vitro transcribed mRNA is purified before it is capped. In some embodiments, the in vitro transcribed mRNA is purified after it is capped. In some embodiments, the in vitro transcribed mRNA is purified before it is tailed. In some embodiments, the in vitro transcribed mRNA is purified after tailing. In some embodiments, the in vitro transcribed mRNA is capped prior to adding the tail. In some embodiments, the capped in vitro transcribed mRNA is purified prior to tailing.

### Optional Tailing Step

In some embodiments, the 3′ tail of the mRNA comprises a poly(A) tail. In some embodiments, the 3′ tail of the mRNA comprises a poly(C) tail. In some embodiments, the tail structure comprises at least 50 adenosine or cytosine nucleotides. In a typical embodiment, the 3′ tail is approximately 100-500 nucleotides in length. For example, a 3′ tail (e.g., a poly(A) tail) of 100-250 nucleotides in length may be particularly useful in therapeutic uses of mRNA.

A poly(A) or poly(C) tail on the 3′ terminus of mRNA typically includes at least 50 adenosine or cytosine nucleotides, at least 100 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, respectively. In some embodiments, a tail structure includes a combination of poly(A) and poly(C) tails with various lengths described herein.

In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

### Optional Capping Step

In vitro transcribed mRNAs with a methylated 5′ cap structure are efficiently translated in vivo. The IVT process can include a cap analogue which is added co-transcriptionally. Alternatively, the 5′ cap structure can be added enzymatically after the IVT reaction has been completed. At least 90% of in vitro transcribed mRNA subjected to enzymatic capping can comprise Cap1 structures.

Several types of 5′ caps are known. A 7-methylguanosine cap (also referred to as “m7G” or “Cap 0”), comprises a guanosine that is linked through a 5′-5′-triphosphate bond to the first transcribed nucleotide. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G (5′) ppp, (5′ (A,G (5′) ppp (5′) A, and G (5′) ppp (5′) G. Additional cap structures are described in U.S. Publication Nos. US 2016/0032356 and US 2018/0125989, which are incorporated herein by reference.

During co-transcriptional capping, a cap analogue is included in the IVT reaction mixture. The cap analogue can be incorporated as the first “base” in a nascent RNA strand. The cap analogue may be Cap 0, Cap 1, Cap 2, m6Am, or a chemical cap analogue. For example, the following chemical cap analogues may be used to generate the 5′-guanosine cap structure according to the manufacturer's instructions: 3′-O-Me-m7G (5′) ppp (5′) G (the ARCA cap); G (5′) ppp (5′) A; G (5′) ppp (5′) G; m7G (5′) ppp (5′) A; m7G (5′) ppp (5′) G; m7G (5′) ppp (5′) (2′OMeA)-pG; m7G (5′) ppp (5′) (2′OmeA) pU; m7G (5′) ppp (5′) (2′OmeG) pG (New England BioLabs, Ipswich, MA; TriLink Biotechnologies).

A vaccinia virus capping enzyme may be used to generate the Cap 0 structure: m7G (5′) ppp (5′) G. A Cap 1 structure may be generated using both vaccinia virus capping enzyme and a 2′-O methyl-transferase to generate: m7G (5′) ppp (5′) G-2′-O-methyl. A Cap 2 structure may be generated from the Cap 1 structure followed by the 2′-O-methylation of the 5′-antepenultimate nucleotide using a 2′-O methyltransferase. A Cap 3 structure may be generated from the Cap 2 structure followed by the 2′-O-methylation of the 5′-preantepenultimate nucleotide using a 2′-O methyltransferase.

In some embodiments, a method in accordance with the invention further comprises a step of capping the in vitro transcribed mRNA. The capping step may involve adding a capping enzyme (guanylyltransferase) and a guanine. A suitable capping enzyme may be derived from a Vaccinia virus (Vaccine virus guanylyltransferase). Typically, the capping step also comprises adding a guanine methyltransferase and a 2′-O-methyltransferase. Capping may be performed separately, e.g., after IVT. The capping step is commonly performed prior to the tailing of the in vitro transcribed mRNA.

In a specific embodiment, the in vitro transcribed mRNA may comprise a 5′ cap with the following structure:

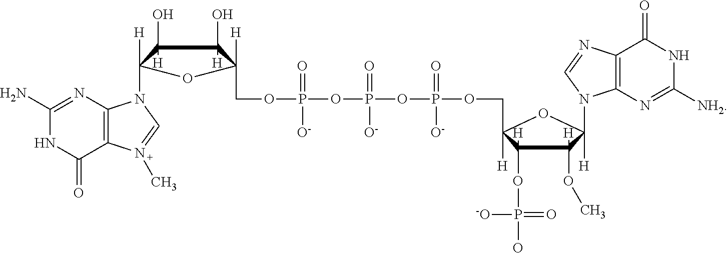

### Purification

The inventors of the present invention have surprisingly discovered that the use of Klebsiella phage KP34 RNA polymerase reduces or eliminates the presence of transcriptional by-products, particularly dsRNA, in in vitro synthesized mRNA. Thus, the mRNA transcripts produced with a method of the invention can be used without the need for further post-purification steps to remove dsRNA. This simplifies the post-synthesis processing of the in vitro synthesized mRNA greatly.

In some embodiments, a method of the invention further comprises a step of purifying the mRNA transcripts obtained from the IVT reaction performed from the KP34 RNA polymerase. In some embodiments, the mRNA transcripts prepared with a method of the invention do not require a purification step to remove dsRNA contaminants. In some embodiments, the step of purifying the mRNA transcripts involves a method other than cellulose chromatography. In some embodiments, the step of purifying the mRNA transcripts involves a method other than HPLC. In some embodiments, the step of purifying the mRNA transcripts involves a method other than HPLC with a buffer system comprising triethylammonium acetate and/or acetonitrile. In some embodiments, the step of purifying the mRNA transcripts involves a method other than anion-exchange fast performance liquid chromatography.

In some embodiments, the mRNA transcripts are purified without a chaotropic agent. In some embodiments, the mRNA transcripts are purified under non-denaturing conditions. In some embodiments, the mRNA transcripts are purified without use of lithium chloride, sodium chloride, potassium chloride, guanidium chloride, guanidium thiocyanate, guanidium isothiocyanate, ammonium acetate, and combinations thereof.

Various methods may be used to purify mRNA. In some embodiments, the mRNA is purified by precipitation and centrifugation. In some embodiments, the mRNA is purified by filtration using, e.g., Normal Flow Filtration (NFF) or Tangential Flow Filtration (TFF).

Suitable purification methods include those described in published U.S. Application Nos. US 2016/0040154, US 2015/0376220, US 2018/0251755, US 2018/0251754, US 2020/0095571, US 2021/0388338, and US 2021/0002635, and in International Patent Publication No. WO 2022/072836, all of which are incorporated by reference herein.

### Compositions

In some embodiments, the invention relates to a composition prepared by a method of the invention. In some embodiments, a composition prepared in accordance with a method of the invention comprises mRNA (e.g., for expression of a therapeutic polypeptide or protein) and Klebsiella phage KP34 RNA polymerase, wherein the composition comprises less than 1% of dsRNA by weight and less than 10% of the mRNA transcripts by weight are abortive transcripts. In particular embodiments, the composition comprises mRNA transcripts substantially free of non-templated nucleic acids. In some embodiments, Klebsiella phage KP34 RNA polymerase is removed by NFF or TFF.

In some embodiments, the invention provides a composition comprising mRNA transcripts (e.g., for expression of a therapeutic polypeptide or protein), wherein the composition comprises less than 1% of dsRNA by weight wherein less than 10% of the mRNA transcripts by weight are abortive transcripts, and wherein the mRNA transcripts are substantially free of non-templated nucleic acids.

### EQUIVALENTS

Although methods and materials similar or equivalent to those described herein can be used in the practice or testing of the present invention, suitable methods and materials are described below.

### EXAMPLES

The following examples are included for illustrative purposes only and are not intended to limit the scope of the invention.

### Example 1. Purification of Klebsiella Phage KP34 RNA Polymerase

This example illustrates affinity purification of Klebsiella phage KP34 RNA polymerase.

The coding sequence of Klebsiella phage KP34 RNA polymerase was cloned into an expression plasmid. An N-terminal His-tag was added to enable affinity purification of the recombinantly produced enzyme yielding an expression plasmid comprising the coding sequence of SEQ ID NO: 3 operatively linked to an IPTG-inducible promoter. The coding sequence encodes a Klebsiella phage KP34 RNA polymerase with the amino acid sequence set forth in SEQ ID NO: 2. BL21 competent E. coli cells (New England Biolabs™) were transformed with the expression plasmid. Successfully transformed E. coli cells were grown in a suitable growth medium including IPTG to induce expression of the recombinant protein. Once the cells reached stationary phase, the E. coli cells were harvested by centrifugation and lysed.

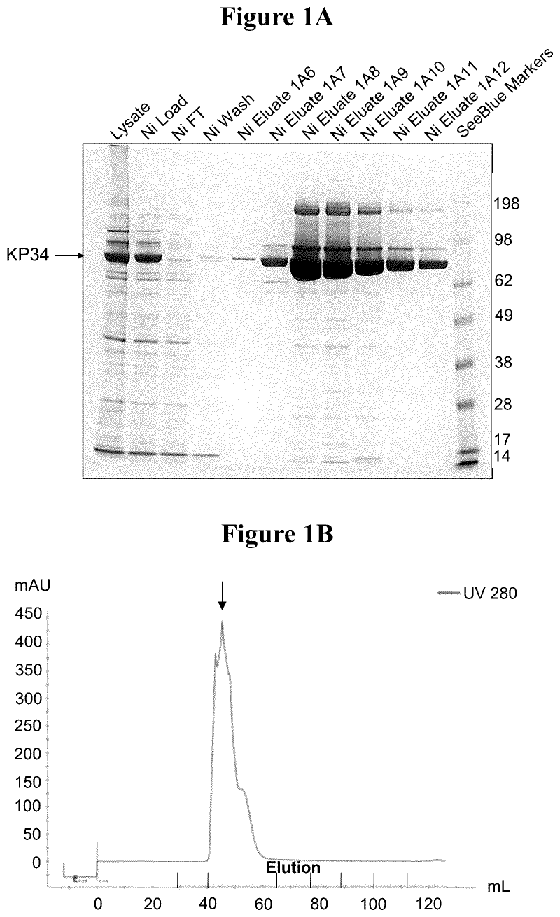

The cell lysates were loaded onto a Ni-NTA agarose column for immobilized metal affinity chromatography (IMAC). After loading, the column was washed to remove any unbound material. To elute the His-tagged Klebsiella phage KP34 RNA polymerase, elution buffer containing increasing concentrations of imidazole were added to the column. The crude lysate, the material loaded on the column, the flow through, wash and various elution fractions were separated on an SDS-PAGE gel to visualize the enzyme (see FIG. 1A). The eluate fractions containing the Klebsiella phage KP34 RNA polymerase were pooled, and the resulting mixture was subjected to size exclusion chromatography (SEC) by gel filtration using a Superdex™ 75 column. The gel-filtered composition included a major peak comprising the polymerase (see FIG. 1B).

This example illustrates that Klebsiella phage KP34 RNA polymerase can be expressed recombinantly in Escherichia coli cells and subsequently purified using affinity chromatography.

### Example 2. Preparing In Vitro Transcribed mRNA

In vitro transcribed mRNA was prepared as described in Example 1 of WO 2021/168052, which is incorporated herein by reference. Briefly, for each gram of mRNA transcribed, a reaction containing a linearized double-stranded DNA plasmid with an RNA polymerase-specific promoter, RNA polymerase (e.g., KP34, SP6, or T7 RNA polymerase, as indicated), RNase inhibitor, pyrophosphatase, NTPs, DTT, and a buffering reagent was prepared with RNase-free water. Unless indicated otherwise, the composition of the reaction buffer was the same for both Klebsiella phage KP34 RNA polymerase and SP6 RNA polymerase (25 mM Tris-HCl, 2 mM spermidine, 25 mM MgCl₂, 0.5 mM NaCl, and pH 7.5). The reaction mixtures were incubated at 37° C. for 60 to 90 min. In vitro transcribed mRNA prepared with ATP, UTP, CTP, and GTP is referred to herein as unmodified (abbreviated as “unmod”). Where indicated, UDP was replaced with N¹-methylpseudouridine to prepare in vitro transcribed mRNA comprising a modified ribonucleotide (abbreviated as “mod”). DNase I was added to stop the reaction, and the reaction mixture was incubated for 15 more minutes at 37° C. The resulting in vitro transcribed mRNA was purified.

### Example 3. In Vitro Transcription

The affinity-purified Klebsiella phage KP34 RNA polymerase obtained in Example 1 was used for IVT as described in Example 2 to assess various parameters such as mRNA yield, integrity of the in vitro transcribed mRNA, non-templated synthesis, or the formation of undesired transcription by-products (double-stranded RNA).

For example, to determine yield and product integrity, a linearized template plasmid encoding a mRNA transcript with a theoretical length of 1944 nucleotides was used (also referred to herein as template no. 1). To assess the occurrence of non-templated additions during IVT, a short 50-nucleotide-long template was used. Where indicated, experiments were performed in parallel to synthesize both unmodified and modified RNA.

### Example 4. Yield and Product Integrity

This example illustrates the use of Klebsiella phage KP34 RNA polymerase in place of SP6 RNA polymerase in the synthesis of both unmodified and modified RNA.

As described in Example 3, a DNA template encoding mRNA transcript with a theoretical length of 1944 nucleotides was contacted with either SP6 RNA polymerase or Klebsiella phage KP34 RNA polymerase. The coding sequence of the mRNA transcript can be used for the expression of a protein. IVT was performed in the presence of ATP, UTP, CTP, and GTP to generate unmodified mRNA (abbreviated as “unmod”). In addition, IVT was also performed with a reaction mixture with a modified ribonucleotide (comprising N¹-methylpseudouridine in place of uridine) to synthesize modified mRNA (abbreviated as “mod”). The results of two independent experiments are summarized in Tables 2 and 3.

The inclusion of a modified ribonucleotide in the reaction mixture did not impact the yield. Notably, the yield achieved with SP6 was on average 3-fold higher than with KP34.

TABLE 2
Comparison of mRNA yield obtained with SP6 and KP34

| | | | | | Total |
|---|---|---|---|---|---|
| | Volume | Concentration | A260/ | A260/ | yield |
| Sample | (mL) | (ng/μL) | 280 | 230 | (mg) |
| SP6 unmod | 1.8 | 866.5 | 2.11 | 2.36 | 2.08 |
| SP6 mod | 1.8 | 857.1 | 1.83 | 2.13 | 2.06 |
| KP34 unmod | 1.5 | 341.5 | 2.11 | 2.31 | 0.68 |
| KP34 mod | 1.8 | 308.6 | 1.82 | 2.08 | 0.74 |

TABLE 3
Comparison of mRNA yield obtained with SP6 and KP34

| | | | | | Total |
|---|---|---|---|---|---|
| | Volume | Concentration | A260/ | A260/ | yield |
| Sample | (mL) | (ng/μL) | 280 | 230 | (mg) |
| SP6 unmod | 1.35 | 1403 | 2.13 | 2.57 | 2.65 |
| SP6 mod | 1.37 | 1317 | 1.85 | 2.28 | 2.4 |
| KP34 unmod | 1.36 | 376.4 | 2.02 | 2.47 | 0.68 |
| KP34 mod | 1.37 | 376.2 | 1.75 | 2.28 | 0.68 |

To determine whether the difference in yield was due to a difference in product integrity, the average length of the mRNA transcripts was analyzed by capillary gel electrophoresis. The results of this analysis are summarized in Tables 4 and 5.

KP34's performance was non-inferior to SP6. About 90% or greater of the mRNA was full-length, with a measured length close to the theoretical value of 1944 nucleotides.

TABLE 4
Comparison of the integrity of mRNA obtained with SP6 and KP34

| | Measured | Relative |
|---|---|---|
| Sample | length, nt | concentration, % |
| SP6 unmod | 1883 | 91.1 |
| SP6 mod | 1797 | 94.5 |
| KP34 unmod | 1875 | 91.9 |
| KP34 mod | 1789 | 92.7 |

TABLE 5
Comparison of the integrity of mRNA obtained with SP6 and KP34

| | Measured | Relative |
|---|---|---|
| Sample | length, nt | concentration, % |
| SP6 unmod | 1958 | 100 |
| SP6 mod | 1875 | 92.2 |
| KP34 unmod | 1925 | 88.7 |
| KP34 mod | 1859 | 90.9 |

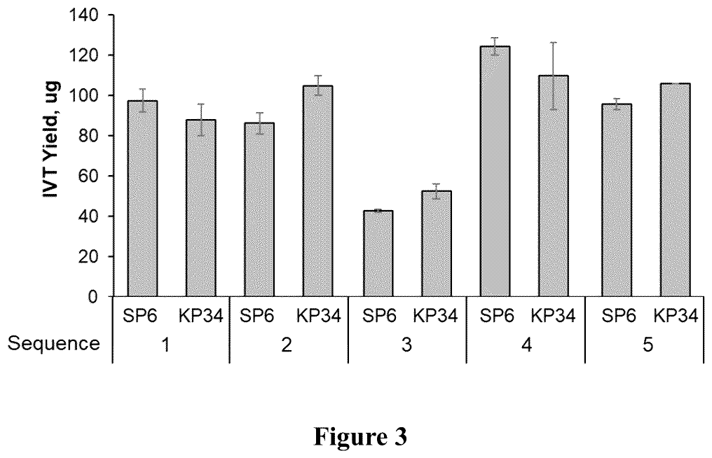

To confirm that non-inferiority in yield was independent of the template used, the experiment was repeated using four DNA templates ranging in size from about 1100 bp to about 4700 bp in addition to the DNA template no. 1, used previously (see Example 3). IVT was performed using either SP6 or KP34 RNA polymerase with a reaction mixture comprising N¹-methylpseudouridine in place of uridine to synthesize modified mRNA. Table 6 shows that the mRNA yield obtained from the IVT reactions is comparable when using either of these RNA polymerases. FIG. 3 illustrates these data with the corresponding standard deviations for each template tested.

TABLE 6
Comparison of mRNA yield obtained with SP6 and KP34

| | |
|---|---|
| Template no. (theoretical | Yield (μg) |

| length of mRNA in bp) | SP6 | KP34 |
|---|---|---|
| 1 (1944 bp), control reaction | 95.7 | 106 |
| 2 (1098 bp) | 97.4 | 87.8 |
| 3 (1712 bp) | 42.8 | 52.4 |
| 4 (1972 bp) | 86.1 | 104.9 |
| 5 (4727 bp) | 124.4 | 109.7 |

Capillary gel electrophoresis was performed on the IVT products to analyze mRNA transcript length. The integrity of mRNA obtained with SP6 or KP34 RNA polymerase was comparable (data not shown).

This example illustrates the use of Klebsiella phage KP34 RNA polymerase in place of SP6 RNA polymerase in the synthesis of both unmodified and modified RNA. About 90% or greater of the resulting transcripts were full-length.

### Example 5. Non-Templated Transcription

This example illustrates that RNA-dependent 3′ end extension of mRNA transcripts does not occur when Klebsiella phage KP34 RNA polymerase is used for IVT.

RNA-dependent 3′ end extension was measured using an RNA-dependent RNA polymerase (RdRp) assay. The template was a 50 base long synthetic RNA (RNA50). This template was allowed to self-anneal. Annealing of an internal region of complementarity results in the formation of a stretch of double-stranded RNA in cis by looping, enabling self-templated 3′end extension.

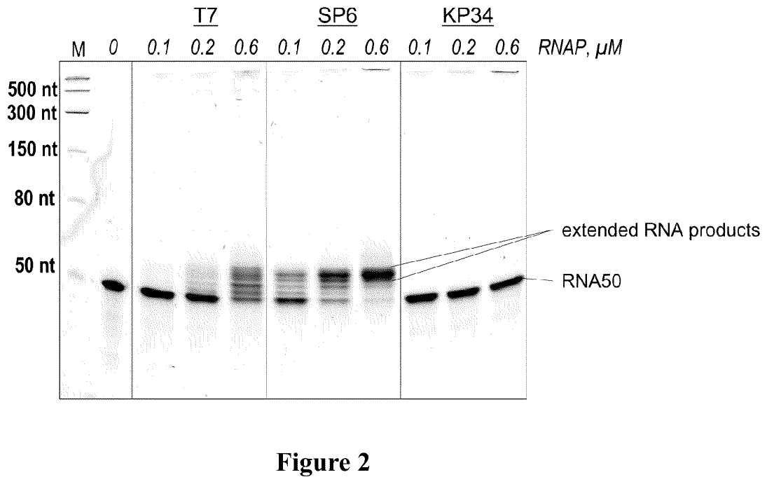

0.4 μM RNA50 template was incubated in the presence of 0 μM (negative control), 0.1 μM, 0.2 μM, or 0.6 μM of either T7, SP6 or KP34 RNA polymerase and unmodified ribonucleotides. Pyrophosphatase and RNase inhibitors were also included in each sample. The samples were incubated for 80 minutes at 37° C. and analyzed by gel electrophoresis on a 15% urea TBE polyacrylamide gel. RNA was detected using SYBR Gold staining. The results are summarized in FIG. 2.

For both T7 and SP6 RNA polymerases, increasing the polymerase concentration increased RNA-dependent 3′ end extension of the RNA template. In contrast, regardless of the tested polymerase concentration, no RNA-dependent 3′end extension was detected during IVT with KP34 RNA polymerase.

To confirm whether the observations with short synthetic RNA templates would apply to longer templates as well, a short 15-base DNA template and a long 1944-base DNA template were amplified side-by-side in separate reactions using either SP6 or KP34 RNA polymerase. The occurrence of non-templated additions of nucleic acids to the 3′ end was determined using liquid chromatography-mass spectrometry (LC-MS). 100 μM of a probe oligonucleotide was annealed to the 3′ end of the mRNA transcripts (1~2 mg RNA per mL, 21 μL in total) in a thermocycler (Eppendorf) at 75° C. for 10 minutes. The samples were then ramped down to 23° C. for 10 minutes and then quickly cooled to 4° C. RNase H nuclease (5000 units/mL, 1 μL, New England BioLabs) and rSAP (1000 units/mL, 4 μL, New England BioLabs) in 1×RNase H buffer (New England BioLabs) was added to each sample and incubated in a thermocycler at 37° C. for 40 minutes.

Ultra-high pressure liquid chromatography (UHPLC) was used for separation of the samples on an Agilent 1290 Infinity II coupled with Agilent InfinityLab C18 2.1×100 mm, 2.7 μm, 100 Å column. The column was heated to 50° C. with a flow rate of 500 μL/min. The mobile phase included buffer A (100 mM HFIP 8.6 mM TEA) and buffer B (100% methanol). The gradient started at 1% buffer B and increased to 5% buffer B for the first 3 minutes, followed by a linear ramp to 20% buffer B until 13 minutes. Between 13 and 14 minutes, the percentage of buffer B increased from 20% to 50%. At 14 minutes, a 1.5-min rinse at 50% buffer B began, followed by a return to 1% of buffer B at 17 minutes.

In this experiment, no non-templated additions of nucleic acids were observed with the short template when KP34 RNA polymerase was used in the synthesis reaction. In contrast, SP6 RNA polymerase produced a heterogenous synthesis product. A portion of the synthesized RNAs included additional non-templated nucleic acids at the 3′ end. Similarly, for the long template, the RNA synthesized with SP6 RNA polymerase was heterogenous in size, with only 59.3% of the RNA having the expected 3′ terminal sequence. In contrast, with KP34 RNA polymerase, the RNA was of a homogenous size, with a 3′ homogeneity of 98%.

This example indicates that RNA-dependent 3′ end extension of mRNA transcripts does not occur when Klebsiella phage KP34 RNA polymerase is used for IVT, even at high concentrations of the polymerase. The example also confirms that KP34 RNA polymerase achieves similar 3′ homogeneity with long DNA templates. In this respect, KP34 RNA polymerase outperforms both T7 and SP6, making it a highly attractive RNA polymerase for the production of high-quality therapeutic RNAs.

### Example 6. Double-Stranded RNA (dsRNA)

This example illustrates that IVT using Klebsiella phage KP34 RNA polymerase yields undetectable amounts of dsRNA.

The process of IVT generates dsRNA through base pairing in regions of complementarity within the same strand or on opposite strands yielding dsRNA with 5′ or 3′ overhangs. In order to measure the amounts of dsRNA generated during IVT using either KP34 or SP6 RNA polymerase, mRNA synthesis was carried out as described in Example 2. 100 ng, 200 ng, or 400 ng RNA in a 2 μL sample volume was blotted on a nitrocellulose membrane. 1 ng, 20 ng, or 40 ng of dsRNA control was used as a reference.

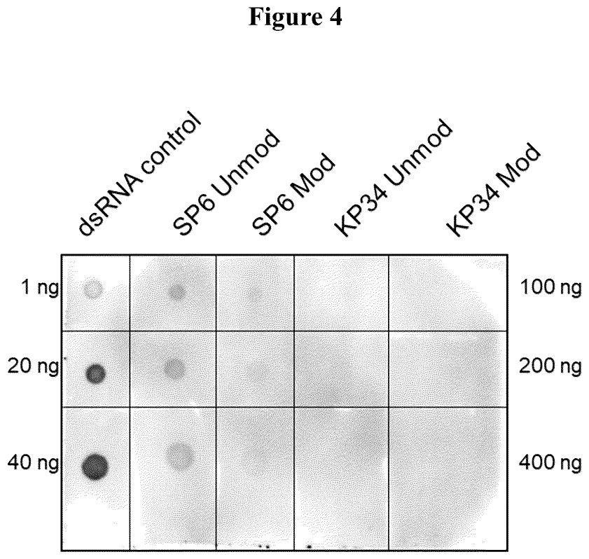

The anti-dsRNA monoclonal antibody J2 was used as the primary antibody. This antibody is the gold standard for the detection of dsRNA. It recognizes dsRNA provided that the length of the helix is greater than or equal to 40 bp. dsRNA-recognition is independent of the sequence and nucleotide composition of the mRNA. An anti-mouse IgG HRP was used as secondary antibody. Signal was detected after a one-minute exposure. The results are shown in FIG. 4.

No dsRNA was detectable with both unmodified and modified RNA transcripts synthesized with Klebsiella phage KP34 RNA polymerase. The presence of dsRNA was detectable in both unmodified and modified RNA transcripts synthesized with SP6 RNA polymerase, with a lower signal detected with modified RNA transcripts.

These results demonstrate that IVT using Klebsiella phage KP34 RNA polymerase generates mRNA transcripts comprising undetectable amounts of dsRNA, as determined by dot blot with the J2 antibody. This example demonstrates that the amount of dsRNA produced by KP34 was many times lower relative to the amount of dsRNA produced by SP6 RNA under IVT conditions that were optimized for SP6.

### Example 7. Improving Activity and Yield

This example illustrates that the activity and yield of Klebsiella phage KP34 RNA polymerase can be increased by removing enzymatically inactive aggregates from the affinity-purified enzyme preparation using size exclusion chromatography (SEC).

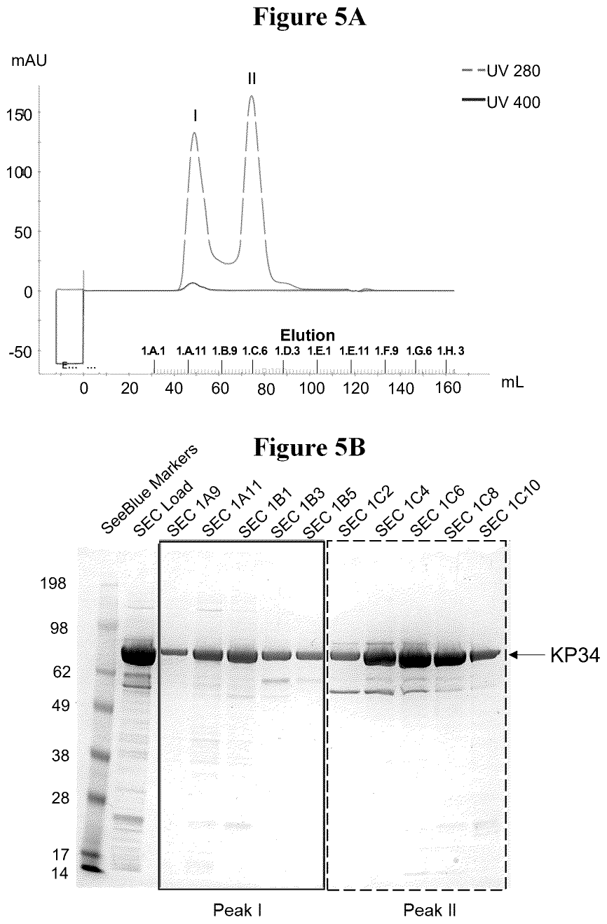

Affinity-purified Klebsiella phage KP34 RNA polymerase was prepared as described in Example 1. Eluate fractions containing the enzyme were pooled and concentrated. The resulting concentrated pool was loaded onto a Superdex™ 200 gel filtration column for size exclusion chromatography with the following buffer: Tris pH 8.0, 200 mM NaCl, 0.1 mM EDTA, 5 mM 2-mercaptoethanol, and 5% glycerol. Under these conditions, two distinct peaks could be resolved (FIG. 5A). Peak I eluted earlier indicating a larger size of the Klebsiella phage KP34 RNA polymerase in this fraction, relative to peak II. Eluates of peak fractions I and II were collected separately. The peak I fraction was estimated to include about 60-70% of the IMAC-purified enzyme.

The collected peak fractions were separated on an SDS-PAGE gel to visualize the Klebsiella phage KP34 RNA polymerase (FIG. 5B). SDS-PAGE gel analysis confirmed that peak I and II fractions included the Klebsiella phage KP34 RNA polymerase. The size of particles in representative samples from each peak was determined by dynamic light scattering (DLS). DLS analysis confirmed that peak fraction I comprised a large proportion of particles with a hydrodynamic diameter of about 50 nm, corresponding to aggregates of the Klebsiella phage KP34 RNA polymerase. In contrast, peak fraction II comprised a large proportion of particles with a hydrodynamic diameter of less than 10 nm.

To determine the enzymatic activity of the two fractions, an in vitro transcription assay was performed with a short template composed of 28 nucleotides comprising a Klebsiella promoter sequence using conditions as described in Example 2. This template yields a 15-nucleotide long RNA transcript. Peak fraction I had almost no enzymatic activity. Based on the hydrodynamic diameter, peak fraction I was comprised of enzymatically inactive aggregates. In contrast, peak fraction II contained the enzymatically active monomeric form of Klebsiella phage KP34 RNA polymerase.

The experiment described in Example 4 was repeated with Klebsiella phage KP34 RNA polymerase from peak fraction II. It was estimated that this fraction comprised less than 5% of enzymatically inactive aggregates. Relative to the experiment described in Example 4, the mRNA yield during in vitro transcription was improved about by 2-fold. The monomeric form of Klebsiella phage KP34 RNA polymerase achieved 70% of the mRNA yield produced by SP6 RNA polymerase as shown in Table 7, below.

TABLE 7
mRNA yield obtained with purified KP34 in which
enzymatically inactive aggregates were removed

| | Concentration | | | Total yield |
|---|---|---|---|---|
| Sample | (ng/μL) | A260/280 | A260/230 | (μg) |
| SP6 Unmod | 1834 | 2.13 | 2.57 | 128 |
| SP6 Mod | 1754 | 1.85 | 2.28 | 123 |
| KP34 Unmod | 1247 | 2.02 | 2.47 | 87 |
| KP34 Mod | 1224 | 1.75 | 2.28 | 86 |

The results in Table 7 demonstrate that the purification of Klebsiella phage KP34 RNA polymerase to remove enzymatically inactive aggregates results in enzyme preparations with improved polymerase activity and mRNA yield.

### Example 8. Purification of KP34 Polymerase by Hydrophobic Interaction Chromatography

Hydrophobic interaction chromatography (HIC) was tested as an alternative purification method to size exclusion chromatography (SEC). Affinity-purified Klebsiella phage KP34 RNA polymerase was prepared as described in Example 1. Several different HIC columns comprising different resins were tested by loading them with the eluate from the Ni²⁺ column. These included Capto phenyl High Sub, Capto phenyl ImpRes, Capto butyl, Capto butyl ImRes and Capto octyl, all in a high-flow agarose matrix. HIC was successfully used to purify KP34. Notably, HIC could be used to purify KP34 without the need for a gel filtration step.

### Example 9. Sequence Optimization Improves Klebsiella Phage KP34 Activity

This example illustrates that mRNA yield of in vitro synthesis reactions employing Klebsiella phage KP34 RNA polymerase can be increased by modifying the nucleic acid sequence 3′ adjacent to the core promoter.

The inventors found that the nucleic acid sequence 3′ adjacent to a core promoter (SEQ ID NO: 4) can promote KP34 RNA polymerase activity. Table 8 shows the core promoter region (bold) and nucleic acid sequence 3′ adjacent to the core promoter, including the partial sequence of a 5′ UTR. Various 3′ adjacent sequences were evaluated (underlined). In SEQ ID NO: 22, the core promoter was followed by a stretch of five Gs and an A. In SEQ ID NOs: 23, 24 and 26, the stretch of Gs immediately 3′ adjacent to the core promoter was shortened by 1, 2, or 3 residues relative to SEQ ID NO: 22. SEQ ID NO: 25 is similar to SEQ ID NO: 24, but additionally lacks the A that is 3′ adjacent to the stretch of Gs. SEQ ID NO: 27 is identical to SEQ ID NO: 22, except that a T was replaced by an A, as indicated.

TABLE 8
KP34 promoter sequences

| SEQ ID NO | Nucleic acid sequence |
|---|---|
| 22 | TAATGTTACAGGAGTAGGGGGACAGATCGCCTGGAGACGC |
| 19 or 23 | TAATGTTACAGGAGTAGGGGACAGATCGCCTGGAGACGC |
| 20 or 24 | TAATGTTACAGGAGTAGGGACAGATCGCCTGGAGACGC |
| 25 | TAATGTTACAGGAGTAGGGCAGATCGCCTGGAGACGC |
| 26 | TAATGTTACAGGAGTAGGACAGATCGCCTGGAGACGC |
| 27 | TAATGTTACAGGAGTAGGGGGACAGAACGCCTGGAGACGC |

IVT was performed as described in Example 3 using KP34 with a DNA template comprising one of the KP34 promoter sequences listed in Table 8. The IVT reaction conditions were those described in Example 2. KP34 was purified by gel filtration to remove enzymatically inactive aggregates. An IVT reaction performed with SP6 RNA polymerase and template plasmid comprising an SP6 promoter served as control. Table 9 shows the mRNA yield of each reaction expressed as a percentage relative to the yield obtained with SP6.

TABLE 9
mRNA yield from template plasmids
with modified KP34 promoters

| Sample | Concentration | | Total yield |
|---|---|---|---|
| (promoter sequence) | (ng/μL) | A260/280 | (% relative to SP6) |
| SP6 Control | 1572 | 1.76 | 100 |
| KP34 #0 | 1387 | 1.77 | 88.2 |
| (SEQ ID NO: 22) | | | |
| KP34 #1 | 1729 | 1.74 | 110 |
| (SEQ ID NO: 23) | | | |
| KP34 #2 | 1633 | 1.70 | 103.6 |
| (SEQ ID NO: 24) | | | |
| KP34 #3 | 923 | 1.71 | 58.6 |
| (SEQ ID NO: 25) | | | |
| KP34 #4 | 1687 | 1.82 | 107.3 |
| (SEQ ID NO: 26) | | | |
| KP34 #5 | 1578 | 1.74 | 100 |
| (SEQ ID NO: 27) | | | |

These results demonstrate that mRNA yield of in vitro synthesis reactions employing Klebsiella phage KP34 RNA polymerase can be increased by modifying the nucleic acid sequence 3′ adjacent to the core promoter. mRNA yield from an IVT reaction employing Klebsiella phage KP34 RNA polymerase can be comparable to an IVT reaction that employs SP6 RNA polymerase. Interestingly, Klebsiella phage KP34 RNA polymerase is more versatile than either SP6 or T7 RNA polymerase. Unlike SP6 or T7, it can utilize templates having a stretch of 4 or 5 Gs in the nucleic acid sequence 3′ adjacent to the core promoter.

### Example 10. Modification of Promoter Adjacent Sequences Improves RNA Yield

This example confirms that modifications made to the nucleic acid sequence 3′ adjacent to the core promoter results in an increased yield when Klebsiella phage KP34 RNA polymerase is used for the in vitro synthesis of mRNA.

In light of the results summarized in Example 9, additional promoter sequences were tested to determine whether mRNA yield could be improved further. Specifically, variants of previously tested promoters were generated. The additional promoter sequences are listed in Table 10.

TABLE 10
Additional promoter sequences

| SEQ ID NO | Nucleic acid sequence |
|---|---|
| 30 | TAATACGACTCACTATAGGACAGATCGCCTGGAGACGC |
| 31 | ATTTAGGTGACACTATAGGACAGATCGCCTGGAGACGC |
| 32 | TAATGTTACAGGAGTAGGGGACAAGATCGCCTGGAGACGC |
| 33 | TAATGTTACAGGAGTAGGAGACAGATCGCCTGGAGACGC |
| 34 | TAATGTTACAGGAGTAGGAGAGAGATCGCCTGGAGACGC |
| 35 | TAATGTTACAGGAGTAGGAGACAGTTTGCCTGGAGACGC |

SEQ ID NOs: 32-35 are variants of the well-performing promoter sequences of SEQ ID NOs: 23 and 26 shown in Table 8. SEQ ID NO: 32 corresponds to SEQ ID NO: 23 with an additional A, as indicated in bold and underlined. SEQ ID NOs: 33-35 correspond to SEQ ID NO: 26 with an additional GA, as indicated in bold and underlined. In SEQ ID NO: 34, a C is additionally replaced with a G, and in SEQ ID NO: 35, two nucleotides are additionally replaced with two Ts. The additional changes are also shown in bold and underlined. The underlining is as shown in Table 8. All modifications were in the nucleic acid sequence 3′ adjacent to the core promoter (SEQ ID NO: 4 in SEQ ID NOs: 32-35, shown in bold) and downstream of the transcription start site. SEQ ID NOs: 30 and 31 are T7 and SP6 promoter sequences, respectively (the nucleic acid sequence 3′ adjacent to the core promoter marked in bold is denoted in SEQ ID NOs: 28 and 29).

IVT was performed as described in Example 3 using KP34 with a DNA template comprising each of the KP34 promoter sequences listed in Tables 8 and 10. IVT was performed with a reaction mixture with a modified ribonucleotide (comprising N¹-methylpseudouridine in place of uridine) to synthesize modified mRNA. Previously tested promoter sequences were included for comparison. The IVT reaction conditions were those described in Example 2.

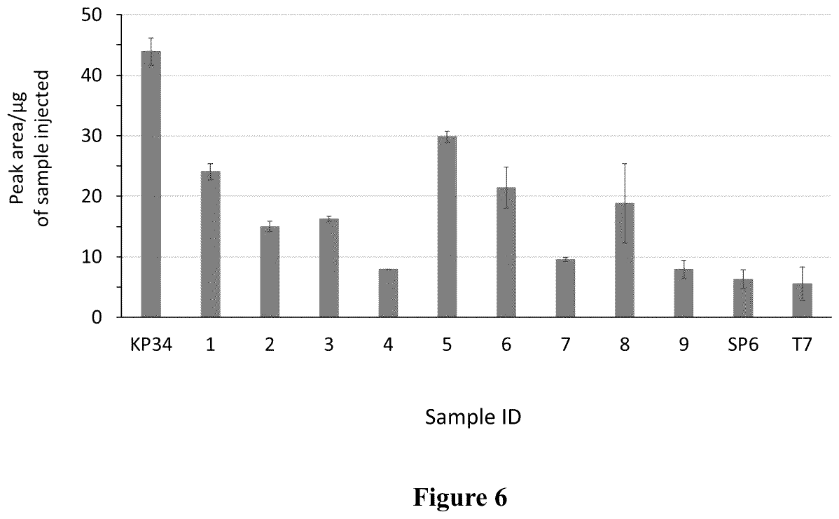

The mRNA yields achieved per reaction with each of the tested promoter sequences are summarized in Table 11. The same sample IDs used in FIG. 6 are as described in Example 11.

TABLE 11
mRNA yield from template plasmids
with the additional test promoters

| | | Yield (μg ± |
|---|---|---|
| | Promoter | standard |
| Sample ID | sequence | deviation) |
| KP34 | SEQ ID NO: 22 | 129.3 ± 28.3 |
| 1 | SEQ ID NO: 23 | 131.2 ± 6.3 |
| 2 | SEQ ID NO: 24 | 148.4 ± 14.6 |
| 3 | SEQ ID NO: 25 | 119.0 ± 2.9 |
| 4 | SEQ ID NO: 26 | 171.4 ± 21.6 |
| 5 | SEQ ID NO: 27 | 137.7 ± 30.6 |
| 6 | SEQ ID NO: 32 | 181.4 ± 35.7 |
| 7 | SEQ ID NO: 33 | 137.7 ± 10.0 |
| 8 | SEQ ID NO: 34 | 164.0 ± 39.6 |
| 9 | SEQ ID NO: 35 | 130.7 ± 3.3 |
| T7 | SEQ ID NO: 30 | 162.8 ± 3.8 |
| SP6 | SEQ ID NO: 31 | 171.1 ± 52.6 |

In this experiment, templates with the promoter sequences of SEQ ID NO: 26 and 32 resulted in the highest yields. The yields were even higher than the yields achieved with the T7 and SP6 RNA polymerases in this experiment.

The results in this example underline the effect of modifications made to the nucleic acid sequence 3′ adjacent to the KP34 core promoter on the mRNA yields that can be achieved with Klebsiella phage KP34 RNA polymerase.

### Example 11. Promoter Optimization Reduces the Amount of Short Abortive Transcripts

This example illustrates that the amount of short abortive transcripts produced by Klebsiella phage KP34 RNA polymerase during an IVT reaction can be reduced by selecting an appropriate promoter sequence.

To determine the impact of the core promoter and the 3′ adjacent nucleic acid sequence on the initiation phase of transcription, the amount of short abortive transcripts in the IVT reactions described in Example 10 was measured using liquid chromatography-mass spectrometry (LC-MS). 100 UM of a probe oligonucleotide was annealed to the 3′ end of the mRNA transcripts (1~2 mg RNA per mL, 21 μL in total) in a thermocycler (Eppendorf) at 75° C. for 10 minutes. The samples were then ramped down to 23° C. for 10 minutes and then quickly cooled to 4° C. RNase H nuclease (5000 units/mL, 1 μL, New England BioLabs) and rSAP (1000 units/mL, 4 μL, New England BioLabs) in 1×RNase H buffer (New England BioLabs) was added to each sample and incubated in a thermocycler at 37° C. for 40 minutes.

Ultra-high pressure liquid chromatography (UHPLC) was used for separation of the samples on an Agilent 1290 Infinity II coupled with Agilent InfinityLab C18 2.1×100 mm, 2.7 μm, 100 Å column. The column was heated to 50° C. with a flow rate of 500 μL/min. The mobile phase included buffer A (100 mM HFIP 8.6 mM TEA) and buffer B (100% methanol). The gradient started at 1% buffer B and increased to 5% buffer B for the first 3 minutes, followed by a linear ramp to 20% buffer B until 13 minutes. Between 13 and 14 minutes, the percentage of buffer B increased from 20% to 50%. At 14 minutes, a 1.5-min rinse at 50% buffer B began, followed by a return to 1% of buffer B at 17 minutes.

The results of this experiment are summarized in the bar graph shown in FIG. 6, which shows the peak area/μg of sample injected. 1 μg of each sample listed in Table 10 was injected into LC-MS. The results in FIG. 6 correspond to the sum of all peaks between 6.5 and 12 mins (n=2).

IVT reactions performed with DNA templates including the promoter sequences of SEQ ID NOs: 26 and 35 resulted in amounts of abortive transcripts that were similar to those that were observed when DNA templates with an SP6 promoter or a T7 promoter were used with their respective RNA polymerase. Surprisingly, using either of the promoter sequences of SEQ ID NOs: 26 or 35, the amounts of abortive transcripts that could be detected in this assay were reduced by about 80% relative to the KP34 promoter sequence of SEQ ID NO: 22. Amounts of abortive transcripts that could be detected using the promoter sequence of SEQ ID NO: 33 were also reduced.

This example illustrates that the amount of short abortive transcripts produced by Klebsiella phage KP34 RNA polymerase during an IVT reaction can be reduced by selecting an appropriate promoter sequence. Notably, the use of DNA templates comprising optimized KP34 promoter sequences resulted in a significant reduction in amount of short abortive transcripts.

### Example 12. KP34-Solubility Tag Fusion Proteins can Increase mRNA Yield

This example illustrates that fusion of the Klebsiella phage KP34 RNA polymerase to a solubility tag can further increase mRNA yield.

To improve solubility and yield of Klebsiella phage KP34 RNA polymerase during recombinant expression in E. coli, a screen of 22 solubility tags was performed. Tags were fused to the N-terminus of the Klebsiella phage KP34 RNA polymerase. The resulting fusion proteins were expressed in E. coli using a 96-well plate format. Each solubility tag was tested in four replicates. E. coli cultures were incubated at 30° C. for 20 hours.

At the end of the incubation period, the cells were lysed. The supernatant was clarified by centrifugation and filtered with a 0.2 μm filter. The fusion proteins were purified by IMAC as described above. The resulting purified protein was desalted using buffer exchange. The protein concentration after purification was quantified using adsorption at 280 nm. Protein amounts were normalized prior to use in IVT reactions.

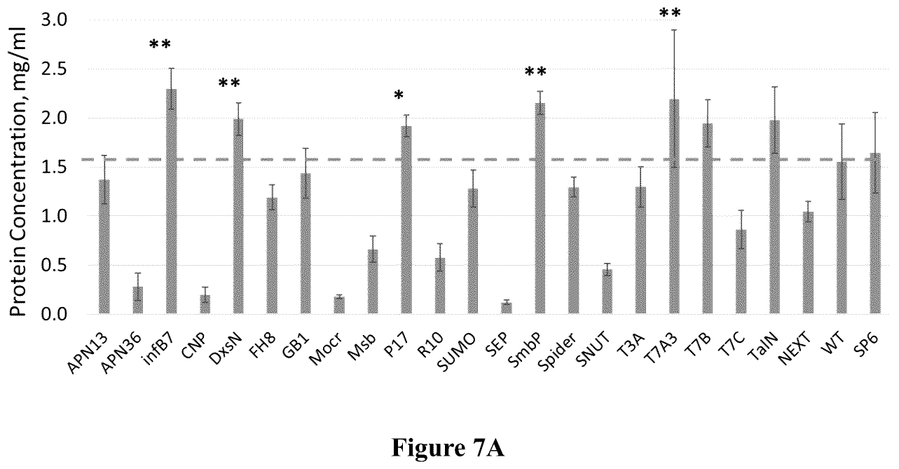

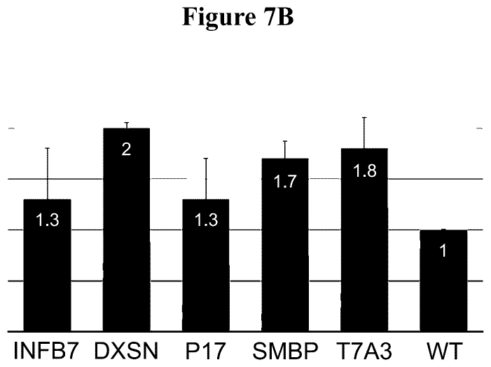

The results of the solubility tag screen are summarized in FIGS. 6A and 6B. The following solubility tags were tested: APN13, APN36, InfB7, CNP, DxsN, Fh8, GB1, Mocr, Msb, P17, R10, SUMO, SEP, SmbP, Spider, SNUT, T3A, T7A3, T7B, T7C, TalN, and NEXT. Each fusion protein was compared to wild-type Klebsiella phage KP34 RNA polymerase (WT) and SP6 expressed and purified using the same conditions. As can be seen from FIG. 7A, fusion to InfB7 (322 μg/mL culture), DxsN (280 μg/mL culture), P17 (266 μg/mL culture), SmbP (308 μg/mL culture), or T7A3 (308 μg/mL culture) resulted in the highest total yield compared to WT (224 μg/mL culture).

The fold-increase in solubility relative to WT is shown in FIG. 7B. Fusion of Klebsiella phage KP34 RNA polymerase to SmbP, T7A3, or DxsN resulted in an about 2-fold increase in solubility (1.7-fold, 1.8-fold, and 2-fold increase, respectively), whereas fusion to InfB7 or P17 resulted in an about 1.3-fold increase.

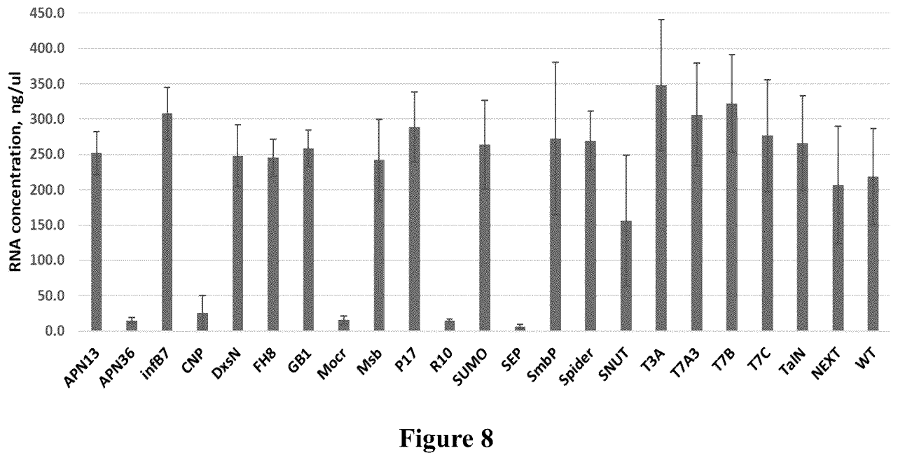

The results of the IVT reactions are summarized in FIG. 8. Increased yield and solubility of the fusion protein was generally mirrored by an increase in mRNA yield. Fusion of Klebsiella phage KP34 RNA polymerase to InfB7, DxsN, P17, SmbP, or T7A3, respectively, resulted in a trend towards higher mRNA yields than WT.

1. A method for manufacturing messenger RNA (mRNA) comprising:
a. providing a DNA template comprising a nucleic acid sequence encoding an mRNA transcript for expression of a polypeptide or protein;
b. contacting the DNA template with a Klebsiella phage KP34 RNA polymerase under conditions suitable for in vitro transcription (IVT) of the mRNA transcript.

2. The method of claim 1, wherein the Klebsiella phage KP34 RNA polymerase provided in step (b) is recombinantly expressed in Escherichia coli cells and purified to remove enzymatically inactive aggregates.

3. The method of claim 2, wherein enzymatically inactive aggregates are removed from an affinity-purified preparation comprising the Klebsiella phage KP34 RNA polymerase.

4. The method of claim 3, wherein the enzymatically inactive aggregates are removed by size exclusion chromatography e.g., gel filtration.

5. The method of any one of the preceding claims, wherein the Klebsiella phage KP34 RNA polymerase comprises less than 5% enzymatically inactive aggregates.

6. The method of any one of the preceding claims, wherein the amino acid sequence of the Klebsiella phage KP34 RNA polymerase is at least 90% identical (e.g., at least 95%, at least 96%, at least 97%, at least 98%, at least 99% identical, or identical) to the amino acid sequence of SEQ ID NO: 1.

7. The method of any one of the preceding claims, wherein the Klebsiella phage KP34 RNA polymerase is present at a concentration ranging from 0.01-0.5 mg/mL.

8. The method of any one of the preceding claims, wherein the mRNA transcript comprises at least 500 ribonucleotides.

9. The method of any one of the preceding claims, wherein the amount of double-stranded RNA (dsRNA) comprised in the mRNA transcripts obtained in step (b) is below the limit of detection.

10. The method of claim 9, wherein the presence of dsRNA is determined by dot blot using antibody J2.

11. The method of any one of the preceding claims, wherein the amount of dsRNA generated in step (b) is at least 10-fold lower relative to a corresponding method using SP6 RNA polymerase or T7 RNA polymerase in place of the Klebsiella phage KP34 RNA polymerase.

12. The method of any one of the preceding claims, wherein the mRNA transcripts obtained in step (b) comprise less than 1% of dsRNA by weight.

13. The method of claim 12, wherein the amount of dsRNA is determined by ELISA using antibodies J2 and K1.

14. The method of any one of the preceding claims, wherein 10% by weight or less of the mRNA transcripts obtained in step (b) comprise non-templated nucleic acids.

15. The method of any one of the preceding claims, wherein less than 10% by weight of the mRNA transcripts obtained in step (b) are abortive transcripts.

16. The method of claim 15, wherein the abortive transcripts comprise less than 20 nucleotides.

17. The method of claim 15 or 16, wherein the abortive transcripts are detectable by gel electrophoresis.

18. The method of any one of the preceding claims, wherein the method comprises synthesizing at least 100 mg of mRNA in a single batch.

19. The method of any one of the preceding claims, wherein the DNA template comprises a Klebsiella phage KP34 promoter sequence operably linked to the nucleic acid sequence encoding the mRNA transcript.

20. The method of claim 19, wherein the promoter sequence comprises the nucleic acid sequence set forth in SEQ ID NO: 5.

21. The method of claim 19 or 20, wherein the promoter sequence is optimized to improve mRNA transcript yield.

22. The method of any one of claims 19-21, wherein the yield of the mRNA transcripts obtained in step (b) is comparable to the yield achieved with a corresponding method using SP6 RNA polymerase or T7 RNA polymerase in place of the Klebsiella phage KP34 RNA polymerase.

23. The method of claim 21 or 22, wherein the promoter sequence comprises the nucleic acid sequence set forth in SEQ ID NO: 7, 8, 9, or 41.

24. The method of claim 21 or 22, wherein the promoter sequence comprises the nucleic acid sequence set forth in SEQ ID NO: 26, 32, or 35, e.g., SEQ ID NO: 26.

25. The method of any one of the preceding claims, wherein the DNA template is at a concentration of 0.05 mg/mL to 0.5 mg/mL.

26. The method of any one of the preceding claims, wherein the DNA template is linear or linearized.

27. The method of any one of the preceding claims, wherein IVT takes place in the presence of magnesium chloride (MgCl₂).

28. The method of claim 27, wherein the MgCl₂ concentration is greater than 20 mM.

29. The method of claim 28, wherein the MgCl₂ concentration is about 25 mM.

30. The method of any one of the preceding claims, wherein IVT takes place in the presence of sodium chloride (NaCl).

31. The method of claim 30, wherein the NaCl concentration is less than 20 mM.

32. The method of claim 31, wherein the NaCl concentration is about 0.5 mM.

33. The method of any one of the preceding claims, wherein IVT takes place in the presence of buffering agent.

34. The method of claim 33, wherein the buffering agent is selected from Tris, HEPES, ammonium sulfate, sodium bicarbonate, sodium citrate, sodium acetate, potassium phosphate, and sodium phosphate.

35. The method of claim 34, wherein the buffering agent is Tris-HCl.

36. The method of claim 35, wherein Tris-HCl is present at a concentration of less than 40 mM.

37. The method of claim 36, wherein Tris-HCl is present at a concentration of about 25 mM.

38. The method of any one of the preceding claims, wherein IVT takes place in the presence of unmodified ribonucleotides.

39. The method of any one of claims 1-38, wherein IVT takes place in the presence of a modified ribonucleotide.

40. The method of claim 39, wherein the modified ribonucleotide has a modified nucleoside.

41. The method of claim 40, wherein the modified nucleoside is selected from 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, 5-methylcytidine, 5-methoxyuridine 7-deazaadenosine, 7-deazaguanosine, 8-oxoadenosine, 8-oxoguanosine, O(6)-methylguanine, pseudouridine, N¹-methylpseudouridine, 2-thiouridine, and 2-thiocytidine.

42. The method of claim 41, wherein the modified nucleoside is pseudouridine, N¹-methylpseudouridine, 5-methylcytidine, or 5-methoxyuridine, e.g., wherein the modified nucleoside is N¹-methylpseudouridine.

43. The method of any one of the preceding claims, wherein IVT takes place in the presence of ribonucleotides, wherein each ribonucleotide is present at a concentration of 0.1 mM to 10 mM.

44. The method of any one of the preceding claims, wherein IVT takes place at a pH of 7.0 to 7.7.

45. The method of claim 44, wherein the pH is about 7.5.

46. The method of any one of the preceding claims, wherein IVT takes place at a temperature of 37° C. to 42° C.

47. The method of claim 46, wherein the temperature is 37° C.

48. The method of any one of the preceding claims, wherein IVT takes place over a period of 30 minutes to 6 hours.

49. The method of any one of the preceding claims, wherein IVT is terminated by addition of DNase I and a DNase I buffer.

50. The method of any one of the preceding claims, further comprising a step of purifying the mRNA transcripts obtained in step (b) from the KP34 RNA polymerase.

51. The method of claim 50, wherein the step of purifying the mRNA transcripts involves a method other than (i) cellulose chromatography, and/or (ii) high-performance chromatography (HPLC) with a buffer system comprising triethylammonium acetate and/or acetonitrile.

52. A composition comprising mRNA transcripts and Klebsiella phage KP34 RNA polymerase, wherein the composition comprises less than 1% of dsRNA by weight and wherein less than 10% of the mRNA transcripts by weight are abortive transcripts.

53. The composition of claim 52, wherein said composition is obtainable by the method of any one of claims 1-49.

54. An mRNA obtained by the method of any one of claims 1-51.

55. A pharmaceutical composition comprising the mRNA of claim 54.

56. A method of treating or preventing a disease or disorder in a subject, said method comprising administering the pharmaceutical composition of claim 55 to the subject.


SEQUENCE LISTING (48 sequences)

SEQ ID NO: 1 - protein, 822 aa, Klebsiella phage KP34
MISALSTVVVPEEALVKRQLELEETYKIRGIERARKLITDALQNGGIMNLPMTQRMLTSAYEVAAAAIDEMRNVKAPGIGGKYRRFLRLIPLDVLTTLSLCTMFEAFSVAPGESASRRQTAQAVMSALGRNVQSELLSLQLRNVAPAYMDRVYEYLTERRTKSPTHILRTLRASAENVHYGHEPWTNAQNISVGRLLCAAVFETGLFQWKTGSGNLSMLYPADDVMEAFQQLVESADTVTMKPPMLVPPVQHTTMWDGGYLTPIDNRGTYHNSHIDRTRLREVAEAFKSADGIKKALNKAQETPYRINKRILELVQEARALGIGVGMPRSVPEPKPEWYLDGVPKENYTEEELDRFGEWKTRMSLWYSADRKRVSQLRSLLTTLEMAEEFKDEKALYFPTCVDWRYRLYFKSSLHPQGSDLQKALLEFGRGKPLGDRGLFWLKVHVATCFGYDKTLFEDRAAWVDANFAEIEQLTVSPFDCPAFTSADSPWCLLAAAIDLVNAVRSGCPEEHISRIPVAMDATNSGGQHLSALLRDPVGGRLTNLYWEGNDKKADLYMDVKRRTDEKVILDLDKEDFIIQSTYWRENEITRSMTKRPSMTYFYSATVRSCSDYIFEGACAEGYEGTDTNSLWNLSCYLAPRMRAAIEEANPAAAAVMGYLQNLARRVPASQHLQWYTPLGGLVMNRYTQREEVRVRIDCMNLSAVLVHNRDFKTCNKRKAASGIAPNFVHSLDSTHLMMVLCAAEGLDIVPIHDSLATHAADVDDMHRHIREQFVRLYEENDLLGDITRAAAAAGADLTDLDMPEVGTLDIRQVLESPFFFC

SEQ ID NO: 2 - protein, 831 aa, synthetic construct
MGHHHHHHGGISALSTVVVPEEALVKRQLELEETYKIRGIERARKLITDALQNGGIMNLPMTQRMLTSAYEVAAAAIDEMRNVKAPGIGGKYRRFLRLIPLDVLTTLSLCTMFEAFSVAPGESASRRQTAQAVMSALGRNVQSELLSLQLRNVAPAYMDRVYEYLTERRTKSPTHILRTLRASAENVHYGHEPWTNAQNISVGRLLCAAVFETGLFQWKTGSGNLSMLYPADDVMEAFQQLVESADTVTMKPPMLVPPVQHTTMWDGGYLTPIDNRGTYHNSHIDRTRLREVAEAFKSADGIKKALNKAQETPYRINKRILELVQEARALGIGVGMPRSVPEPKPEWYLDGVPKENYTEEELDRFGEWKTRMSLWYSADRKRVSQLRSLLTTLEMAEEFKDEKALYFPTCVDWRYRLYFKSSLHPQGSDLQKALLEFGRGKPLGDRGLFWLKVHVATCFGYDKTLFEDRAAWVDANFAEIEQLTVSPFDCPAFTSADSPWCLLAAAIDLVNAVRSGCPEEHISRIPVAMDATNSGGQHLSALLRDPVGGRLTNLYWEGNDKKADLYMDVKRRTDEKVILDLDKEDFIIQSTYWRENEITRSMTKRPSMTYFYSATVRSCSDYIFEGACAEGYEGTDTNSLWNLSCYLAPRMRAAIEEANPAAAAVMGYLQNLARRVPASQHLQWYTPLGGLVMNRYTQREEVRVRIDCMNLSAVLVHNRDFKTCNKRKAASGIAPNFVHSLDSTHLMMVLCAAEGLDIVPIHDSLATHAADVDDMHRHIREQFVRLYEENDLLGDITRAAAAAGADLTDLDMPEVGTLDIRQVLESPFFFC

SEQ ID NO: 3 - DNA, 2493 bp, synthetic construct
  source 1..2493: mol_type = other DNA
atgggacatcaccatcaccatcacggaggcattagcgccctaagtacggtagtagtaccagaggaagcactggtgaaacgccagctggagcttgaagagacctataagattcgcggaatcgagcgggcacgtaagctgattacggacgcattgcagaacggcgggattatgaacctgcctatgacgcagcgtatgctcacttcggcatatgaagtggctgctgccgcgatcgatgagatgcgaaatgtcaaagcccctggcattggtgggaagtatcgccggttcctgcgcttaatccccttggatgtcctgaccaccctgagcctgtgcacaatgtttgaggcgttcagcgtcgcccccggcgagtccgccagtcgccgccagactgcgcaggcggtaatgtccgcactgggccggaacgtgcagtcggaactgctgtctttgcagctgcgtaacgttgccccggcgtacatggaccgggtgtatgagtatctcacagagcgccgtacaaagtcacctacgcacatcctgcgtacgctccgtgccagtgccgagaacgtgcactacgggcacgagccttggaccaatgcccagaacatctccgtagggcgtctgctgtgtgccgcagtattcgagacgggtctgttccagtggaagacgggcagcgggaacctgagcatgctctaccccgcagacgacgttatggaagcatttcagcagctagtggaatctgctgacactgtgacgatgaagcctcctatgctggtgccgccggtgcagcacactacgatgtgggacggcgggtacctcaccccaatcgataaccgcggtacgtaccataactcgcacatcgaccgcaccagactgcgtgaagtagcggaagcatttaagtctgcggacggcatcaagaaggcgcttaataaggcacaggaaactccgtaccgtattaataagcgcatactggaactggtgcaagaagcacgggccctaggtatcggagtgggtatgccccgctcagtgccggagcctaaaccggagtggtacttggatggcgtaccaaaagagaactacacagaggaagaactggaccgcttcggtgagtggaagacgcgtatgtctctgtggtacagtgctgaccgtaagcgtgtgtcgcaactgcgcagccttctgactacgttagaaatggcagaggaattcaaagatgagaaagctctttacttcccgacttgtgtggactggcgctaccgcctgtacttcaagtcctcactgcatccccaaggttctgatttgcagaaagcccttcttgagtttggcagaggaaaacctctgggtgatcgcggcttattctggctcaaggtgcatgtcgccacatgctttggttatgacaagaccttattcgaagaccgcgcagcttgggttgatgcgaactttgcagagattgagcagcttacagtttcaccgtttgattgccctgctttcacctccgcggacagcccttggtgcttattggccgccgctatcgacctggttaatgctgtgcgttctggatgcccagaagagcatattagccgaatcccagttgctatggacgctacaaactctggtggacagcacctctcagcgctcctgagagaccctgtaggcggtcgtctgacgaacctgtactgggaaggtaacgacaagaaagcggacctgtacatggatgtgaagcgccgtacggacgagaaggtgatactggacctggacaaggaggacttcattatccagagcacgtactggagagagaacgaaatcacccgcagcatgaccaagcgccccagtatgacctacttctacagcgccacggtgcgtagctgcagcgactacatctttgaaggcgcttgcgctgagggatacgagggtaccgatactaacagtctgtggaacctgtcgtgttacctggcaccgcgtatgcgtgccgctatcgaggaggcaaaccccgctgctgcggcagttatggggtacttgcagaacctagctagacgtgtaccggcaagccagcacctgcagtggtatacgccgctgggtgggctcgtaatgaaccgctacacgcagcgtgaagaagtgcgcgtacgtattgactgcatgaacctatcggcggtactggtacacaaccgggacttcaagacctgcaacaagcgtaaggcagcctccgggattgccccgaactttgtgcatagtctggacagtacgcacttgatgatggtgctctgcgctgcggagggtctggatattgtgccgattcacgactcactggctactcacgcagccgacgttgatgatatgcaccgacacatccgcgaacagtttgtgcgcctctacgaggagaatgacctgcttggcgacattactcgcgcggcggcagcagccggggcagacttgacggacctggacatgcctgaggtgggtactttggacatccgtcaagtgctagaatccccgttcttcttctgc

SEQ ID NO: 4 - DNA, 16 bp, synthetic construct
  source 1..16: mol_type = other DNA
taatgttacaggagta

SEQ ID NO: 5 - protein, 14 aa, synthetic construct
ATGTTACAGGAGTA

SEQ ID NO: 6 - DNA, 19 bp, synthetic construct
  source 1..19: mol_type = other DNA
taatgttacaggagtaggg

SEQ ID NO: 7 - DNA, 21 bp, synthetic construct
  source 1..21: mol_type = other DNA
taatgttacaggagtagggga

SEQ ID NO: 8 - DNA, 20 bp, synthetic construct
  source 1..20: mol_type = other DNA
taatgttacaggagtaggga

SEQ ID NO: 9 - DNA, 19 bp, synthetic construct
  source 1..19: mol_type = other DNA
taatgttacaggagtagga

SEQ ID NO: 10 - DNA, 27 bp, synthetic construct
  source 1..27: mol_type = other DNA
taatgttacaggagtagggggannnnw

SEQ ID NO: 11 - DNA, 27 bp, synthetic construct
  source 1..27: mol_type = other DNA
taatgttacaggagtagggggacagat

SEQ ID NO: 12 - DNA, 27 bp, synthetic construct
  source 1..27: mol_type = other DNA
taatgttacaggagtagggggacagaa

SEQ ID NO: 13 - DNA, 24 bp, synthetic construct
  source 1..24: mol_type = other DNA
gggggacagatcgcctggagacgc

SEQ ID NO: 14 - DNA, 23 bp, synthetic construct
  source 1..23: mol_type = other DNA
ggggacagatcgcctggagacgc

SEQ ID NO: 15 - DNA, 22 bp, synthetic construct
  source 1..22: mol_type = other DNA
gggacagatcgcctggagacgc

SEQ ID NO: 16 - DNA, 21 bp, synthetic construct
  source 1..21: mol_type = other DNA
gggcagatcgcctggagacgc

SEQ ID NO: 17 - DNA, 21 bp, synthetic construct
  source 1..21: mol_type = other DNA
ggacagatcgcctggagacgc

SEQ ID NO: 18 - DNA, 24 bp, synthetic construct
  source 1..24: mol_type = other DNA
gggggacagaacgcctggagacgc

SEQ ID NO: 19 - DNA, 39 bp, synthetic construct
  source 1..39: mol_type = other DNA
taatgttacaggagtaggggacagatcgcctggagacgc

SEQ ID NO: 20 - DNA, 38 bp, synthetic construct
  source 1..38: mol_type = other DNA
taatgttacaggagtagggacagatcgcctggagacgc

SEQ ID NO: 21 - DNA, 137 bp, synthetic construct
  source 1..137: mol_type = other DNA
cagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 22 - DNA, 40 bp, synthetic construct
  source 1..40: mol_type = other DNA
taatgttacaggagtagggggacagatcgcctggagacgc

SEQ ID NO: 23 - DNA, 39 bp, synthetic construct; identical to SEQ ID NO: 19
  source 1..39: mol_type = other DNA

SEQ ID NO: 24 - DNA, 38 bp, synthetic construct; identical to SEQ ID NO: 20
  source 1..38: mol_type = other DNA

SEQ ID NO: 25 - DNA, 37 bp, synthetic construct
  source 1..37: mol_type = other DNA
taatgttacaggagtagggcagatcgcctggagacgc

SEQ ID NO: 26 - DNA, 37 bp, synthetic construct
  source 1..37: mol_type = other DNA
taatgttacaggagtaggacagatcgcctggagacgc

SEQ ID NO: 27 - DNA, 40 bp, synthetic construct
  source 1..40: mol_type = other DNA
taatgttacaggagtagggggacagaacgcctggagacgc

SEQ ID NO: 28 - DNA, 17 bp, synthetic construct
  source 1..17: mol_type = other DNA
taatacgactcactata

SEQ ID NO: 29 - DNA, 17 bp, synthetic construct
  source 1..17: mol_type = other DNA
atttaggtgacactata

SEQ ID NO: 30 - DNA, 38 bp, synthetic construct
  source 1..38: mol_type = other DNA
taatacgactcactataggacagatcgcctggagacgc

SEQ ID NO: 31 - DNA, 38 bp, synthetic construct
  source 1..38: mol_type = other DNA
atttaggtgacactataggacagatcgcctggagacgc

SEQ ID NO: 32 - DNA, 40 bp, synthetic construct
  source 1..40: mol_type = other DNA
taatgttacaggagtaggggacaagatcgcctggagacgc

SEQ ID NO: 33 - DNA, 39 bp, synthetic construct
  source 1..39: mol_type = other DNA
taatgttacaggagtaggagacagatcgcctggagacgc

SEQ ID NO: 34 - DNA, 39 bp, synthetic construct
  source 1..39: mol_type = other DNA
taatgttacaggagtaggagagagatcgcctggagacgc

SEQ ID NO: 35 - DNA, 39 bp, synthetic construct
  source 1..39: mol_type = other DNA
taatgttacaggagtaggagacagtttgcctggagacgc

SEQ ID NO: 36 - DNA, 25 bp, synthetic construct
  source 1..25: mol_type = other DNA
taatgttacaggagtaggacagatc

SEQ ID NO: 37 - DNA, 12 bp, synthetic construct
  source 1..12: mol_type = other DNA
ggggacaagatc

SEQ ID NO: 38 - DNA, 11 bp, synthetic construct
  source 1..11: mol_type = other DNA
ggagacagatc

SEQ ID NO: 39 - DNA, 11 bp, synthetic construct
  source 1..11: mol_type = other DNA
ggagagagatc

SEQ ID NO: 40 - DNA, 11 bp, synthetic construct
  source 1..11: mol_type = other DNA
ggagacagttt

SEQ ID NO: 41 - DNA, 28 bp, synthetic construct
  source 1..28: mol_type = other DNA
taatgttacaggagtaggggacaagatc

SEQ ID NO: 42 - DNA, 27 bp, synthetic construct
  source 1..27: mol_type = other DNA
taatgttacaggagtaggagacagatc

SEQ ID NO: 43 - DNA, 27 bp, synthetic construct
  source 1..27: mol_type = other DNA
taatgttacaggagtaggagagagatc

SEQ ID NO: 44 - DNA, 27 bp, synthetic construct
  source 1..27: mol_type = other DNA
taatgttacaggagtaggagacagttt

SEQ ID NO: 45 - DNA, 26 bp, synthetic construct
  source 1..26: mol_type = other DNA
atgttacaggagtaggggacaagatc

SEQ ID NO: 46 - DNA, 25 bp, synthetic construct
  source 1..25: mol_type = other DNA
atgttacaggagtaggagacagatc

SEQ ID NO: 47 - DNA, 25 bp, synthetic construct
  source 1..25: mol_type = other DNA
atgttacaggagtaggagagagatc

SEQ ID NO: 48 - DNA, 25 bp, synthetic construct
  source 1..25: mol_type = other DNA
atgttacaggagtaggagacagttt